(19) United States
(12) Patent Application Publication
(10) Pub. No.: US 2025/0250358 A1
(43) Pub. Date: Aug. 7, 2025

(54) DEGRADATION OF SURFACE PROTEINS USING DUAL BINDING AGENT

(71) Applicant: The Regents of the University of California, Oakland, CA (US)

(72) Inventors: Josef A. GRAMESPACHER, San Francisco, CA (US); Adam D. COTTON, San Francisco, CA (US); James A. WELLS, San Francisco, CA (US)

(21) Appl. No.: 18/856,789

(22) PCT Filed: Apr. 14, 2023
(86) PCT No.: PCT/US23/65803

§ 371 Date: Oct. 14, 2024

Related U.S. Application Data
(60) Provisional application No. 63/331,503, filed on Apr. 15, 2022.

(51) Int. Cl. C07K 16/40; C07K 16/28

(52) U.S. Cl. CPC C07K 16/40; C07K 16/2827; C07K 16/2863; C07K 2317/31; C07K 2317/34; C07K 2317/35; C07K 2317/41; C07K 2317/55; C07K 2317/732; C07K 2317/92

(57) ABSTRACT

The present disclosure relates to, among other things, methods for degrading targeted surface proteins using the ubiquitin pathway by using a dual binding agent that binds the targeted surface protein and a membrane-associated ubiquitin E3 ligase. The disclosure also provides compositions and methods useful for producing such dual binding agents, nucleic acids encoding same, host cells genetically modified with the nucleic acids, as well as methods for modulating an activity of a cell and/or for the treatment of various diseases such as cancers.

### CROSS-REFERENCE TO RELATED APPLICATION

The present application claims priority to U.S. Provisional Patent Application Ser. No. 63/331,503, filed on Apr. 15, 2022. The content of the above-referenced application is herein expressly incorporated by reference in its entirety, including any drawings.

### STATEMENT REGARDING FEDERALLY SPONSORED R&D

This invention was made with government support under grants P41 CA196276 and R35 GM122451 awarded by The National Institutes of Health. The government has certain rights in the invention.

### INCORPORATION BY REFERENCE OF SEQUENCE LISTING

The material in the accompanying Sequence Listing is hereby incorporated by reference into this application. The accompanying Sequence Listing file, named 2023 Apr. 14 Sequence_Listing_ST26 048536-732001WO.xml, was created on Apr. 14, 2023, and is 23,846 bytes in size.

### FIELD

The present disclosure relates generally to new methods and agents for degrading surface proteins on a cell using the ubiquitin and/or internalization pathway. The disclosure also provides methods useful for producing such agents, nucleic acids encoding same, host cells genetically modified with the nucleic acids, as well as methods for modulating an activity of a cell and/or for the treatment of various diseases such as cancers.

### BACKGROUND

Targeted protein degradation is a promising new therapeutic strategy compared to conventional inhibition-based therapeutics. Inhibitors rely on sustained, occupancy-driven pharmacology, necessitating high affinity binders capable of abrogating catalytic or binding functions. Inhibiting protein-protein interactions or scaffolding functions has been extremely challenging for standard binding-based small molecules. In contrast, protein degraders are catalytic and utilize event-driven pharmacology, alleviating the need for high affinity binders, and durably abrogate all protein functions at once. As such, degrader technologies such as proteolysis targeting chimeras (PROTACs) have had great success in targeting traditionally challenging proteins. A number of PROTACs are currently in clinical trials.

Most degrader technologies, including PROTACs, utilize an intracellular mechanisms of action and have thus been largely limited to targeting proteins with cytoplasmic domains. However, recent approaches, such as LYTACs have been described for specifically degrading cell surface proteins. These utilize recycling glycan receptors such as the mannose-6-phosphate receptor4 (M6PR) or asialoglycoprotein receptor (ASGR) to target proteins for internalization and trafficking to the lysosome for degradation. These require complex glycans conjugated to antibodies or to small molecules to effect degradation of a membrane protein.

As a hybrid approach that is broadly applicable to many cell types, we recently described antibody-based PROTACs (AbTACs). AbTACs utilize a standard IgG bispecific antibody format to bring a cell surface E3 ligase (RNF43) into proximity of a membrane protein of interest (POI) to mediate its degradation through the lysosomal pathway. The traditional bispecific IgG scaffold on which the AbTAC is built possesses favorable pharmacokinetic properties relative to LYTACS and other small molecule based degraders. Furthermore, in contrast to other degradation modalities such as LYTACS and PROTACS, AbTACs are fully recombinant. However, previous AbTACS have only been able to achieve approximately 60% degradation of target protein.

To solve this problem, the present disclosure provides new AbTACS comprising IgG scaffolds that exhibit increased Dmax as compared to previous AbTACS.

All references and patents cited herein are hereby incorporated by reference in full, as if fully set forth herein.

### SUMMARY

The present disclosure provides a dual binding agent that includes a) a first binding domain that specifically binds to a E3 ligase, wherein the first binding domain comprises an IgG scaffold; and b) a second binding domain that specifically binds to an extracellular epitope on a target protein of a target cell, wherein the second binding domain is fused to the C-terminus of the light chain of the IgG scaffold, and wherein both the E3 ligase and the target protein are membrane associated.

The present disclosure also provides a dual binding agent that includes a) a first binding domain that specifically binds to a E3 ligase, wherein the first binding domain comprises an IgG scaffold; and b) a second binding domain that specifically binds to an extracellular epitope on a target protein of a target cell, wherein the second binding domain is fused to the C-terminus of the heavy chain of the IgG scaffold, and wherein both the E3 ligase and the target protein are membrane associated.

The present disclosure further provides a dual binding agent that includes a) a first binding domain that specifically binds to a E3 ligase, wherein the first binding domain comprises an IgG scaffold; and b) a second binding domain that specifically binds to an extracellular epitope on a target protein of a target cell, wherein the second binding domain is fused to the N-terminus of the heavy chain of the IgG scaffold, and wherein both the E3 ligase and the target protein are membrane associated.

In one embodiment, binding of the dual binding agent to both the E3 ligase and the target protein results in ubiquitination and/or internalization of the target protein.

In one embodiment, the target cell is a neoplastic cell.

In one embodiment, the target protein is selected from the group consisting of PD-L1, PD-1, CTLA-4, A2AR, B7-H3, B7-H4, BTLA, KIR, LAG3, NKG2D, TIM-3, VISTA, and SIGLEC7.

In one embodiment, the first binding domain specifically binds to an extracellular protein attached to an E3 ligase or a transmembrane protein that interacts with an E3 ligase.

In one embodiment, the dual binding agent induces degradation of the target protein with a Dmax of at least 20%.

In one embodiment, degradation of the target protein reduces the ability of the target cell to proliferate.

In one embodiment, the target protein is selected from the group consisting of HER2, CD19, CD20, CDCP1, PD-L1, EGFR, MMP14, and CTLA-4.

In one embodiment, the E3 ligase is a transmembrane protein. In one embodiment, the E3 ligase is selected from the group consisting of RNF43, ZNRF3, RNF133, RNF148, GRAIL (RNF128), RNF149, Goliath (RNF130), RNF150, RNF122, ZNRF4, RNF13, RNF167, RNF121, RNF175, DCST1, March6, Kf-1 (RNF103), RNF182, RNF145, TRC8 (RNF139), HRD1 (SYVN1), RNFT1, MAPL (MUL1), RNF152, RNF26, RINES (RNF180), MARCHF3, MARCHF2, MARCHF8, MARCH1 (MARCHF1), March11, MARCHF9, March4, RNF186, RNF170, RNF185, RMA1 (RNF5), TRIM59 (RNF104), TRIM13, MARCHF5 (MARCH5), RNF197 (CGRF1), RNF183, RNF217, RNF144B, RNF144A, RNF19B, and RNF19A.

In one embodiment, the second binding domain is selected from the group consisting of an sc-Fv, single-domain antibodies, nanobodies, Fabs, monospecific Fab2, Fc, minibodies, IgNAR, V-NAR, hcIgG, VHH domains, camelid antibodies, peptibodies, DARPins, and a small molecule.

The present disclosure also provides a nucleic acid that encodes the dual binding agent of the present disclosure. In one embodiment, the nucleic acid is operably connected to a promoter.

The present disclosure also provides an engineered cell comprising the nucleic acid of the present disclosure. In one embodiment, the cell is a B cell, a B memory cell, or a plasma cell.

The present disclosure also provides a method for making a dual binding agent. The method includes a) introducing into a host cell one or more of the nucleic acid(s) of the present disclosure; b) culturing the host cell of step (a) and c) inducing expression of the dual binding agent.

The present disclosure further provides a vector, comprising the nucleic acid of the present disclosure.

Also provided herein is a pharmaceutical composition that includes (1) the dual binding agent of the present disclosure, the nucleic acid of the present disclosure, and (2) a pharmaceutically acceptable carrier.

The present disclosure further provides a method of treating a neoplastic disease or disorder in a subject. The method includes administering to a subject in need thereof, a therapeutically effective amount of: a) the dual binding agent of the present disclosure; b) the nucleic acid of the present disclosure; or c) the cell of the present disclosure.

The present disclosure also provides a use for the treatment of neoplastic disease of: a) the dual binding agent of the present disclosure; b) the nucleic acid of the present disclosure; or c) the cell of the present disclosure.

Also provided herein is a use for the manufacture of a medicament for the treatment of neoplastic disease of: a) the dual binding agent of the present disclosure; b) the nucleic acid of the present disclosure; or c) the cell of the present disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1C show relative levels of RNF43 and POI affect AbTAC mediated degradation. FIG. 1A is a cartoon depiction of the AbTAC mediated degradation mechanism. FIG. 1B is a table showing RNA transcript levels for RNF43 and PD-L1 in different cell lines and the maximal degradation of PD-L1 achieved in each respective cell line using an R0/Atz AbTAC. (TPM=transcripts per Kilobase million). FIG. 1C is a graph of degradation assays depicting surface levels of PD-L1 as measured by flow cytometry following 24 hr incubation of 10 nM R0/Atz on either WT T24 cells (−), T24 cells overexpressing WT RNF43 (T24 R-WT), or T24 cells overexpressing an RNF43 mutant in which the intracellular domain has been replaced with eGFP (T24 R-MUT). PD-L1 levels are relative to un-treated cells and represent the average of at least three independent biological replicates. Error bars represent one standard deviation of uncertainty. T tests were utilized to determine significance ***=P≤0.001, ns=not significant.

FIG. 2 shows cell surface RNF43 levels of WT and engineered T24 cells. Flow cytometry measurement of the cell surface levels of RNF43 on either T24 WT cells, T24 cells overexpressing WT RNF43 (T24 R-WT), or T24 cells overexpressing an RNF43 mutant in which the intracellular domain has been replaced with eGFP (T24 R-MUT).

FIG. 3 shows SDS-PAGE of AbTACs used in this study. SDS-Polyacrylamide gel electrophoresis (PAGE) analysis of each purified AbTAC utilized in this study.

FIG. 4 shows PD-L1 antibody used in flow-based degradation readout binds separate epitope than Atezolizumab. BLI (Top) and Flow cytometry (Bottom) based analysis indicating that the PD-L1 (D8T4X)-647 antibody used for flow cytometry-based measurement of PD-L1 levels in degradation assays binds a different epitope than Atezolizumab. (TOP) Tips were first incubated with Atezoliumab Fab followed by the indicated Antibody. (Bottom) Cells were first incubated with Atezoliumab Fab followed by the indicated Antibody.

FIG. 5 shows binding kinetics and CDR sequences of Fabs utilized in this study. Bio-layer interferometry kinetic measurements for each of the Fabs utilized in this study along with the respective CDR sequences.

FIG. 6 shows RNF43 Fabs specifically bind RNF43 on cells. RNF43 Clones 0, 3, and 6 specifically bind RNF43 on cells. Fabs were incubated with HEK 293T cells overexpressing either RNF43 (ECD)-eGFP (RNF43 in which the intracellular domain is replaced with eGFP) or ZNRF3 (ECD)-eGFP (ZNRF3 in which the intracellular domain is replaced with eGFP).

FIG. 7 shows RNF43 Fab clones 0, 3, and 6 bind unique epitopes on RNF43. Fabs bind unique epitopes based on additive BLI. For each graph, 100 nM of the first construct was added followed by 100 nM of the first construct and 100 nM of the second construct. Data indicate that the 2^{nd} added Fab binds a distinct epitope from the first added Fab.

FIGS. 8A-8H show epitope and affinity of E3 ligase and POI binding arms affect AbTAC mediated degradation. FIGS. 8A-8C are graphs of degradation assays depicting surface levels of PD-L1 as measured by flow cytometry following 24 hr incubation of 10 nM of the indicated AbTAC on T24 R-WT cells. PD-L1 levels are relative to un-treated cells. FIG. 8A is a graph showing that AbTACs with binding arms that bind different RNF43 epitopes degrade PD-L1 to different levels. FIG. 8B is a graph showing that affinity of RNF43 binding arm is mildly correlated to degradation efficiency. FIG. 8C is a graph showing that affinity of the POI binding arm correlates to degradation efficiency. FIGS. 8B-8C show a linear regression analysis was utilized to determine correlation. FIGS. 8D-8F show degradation assays depicting levels of EGFR as measured by Western blot following 24 hr incubation of 10 nM of the indicated AbTAC on T24 R-WT cells. EGFR levels are relative to un-treated cells. FIG. 8D is a representative Western blot showing that R0/Ctx can degrade EGFR. In FIGS. 8E-8F, Depa=depatuxizumab²¹, Nimo=nimotuzumab²², Matu=matuzumab²³, Neci=necitumumab²⁴, Pani=panitumumab²⁵, Ctx=cetuximab²² FIG. 8E is a graph showing that AbTACs with binding arms that bind different EGFR epitopes degrade EGFR to different levels. FIG. 8F is a graph depicting that affinity of a specific EGFR binder arm does not correlate with degradation efficiency. FIGS. 8G-8H are graphs of degradation assays depicting levels of PD-L1 (FIG. 8G) or EGFR (FIG. 8H) as measured by Western blot following 24 hr incubation of the indicated AbTAC at the indicated concentration on HCC2935 cells. (FIGS. 8A-8C, 8E-8H) PD-L1 and EGFR levels are relative to un-treated cells and represent the average of at least three independent biological replicates. Error bars represent one standard deviation of uncertainty. T tests were utilized to determine significance ***=P≤0.001, **=P≤0.01, *=P≤0.05, ns=not significant.

FIG. 9 shows a representative Western blot of EGFR degradation on T24 R-WT cells. Western blot of degradation assays depicting densitometry levels of EGFR following 24 hr incubation of 10 nM of the indicated AbTAC on T24 R-WT cells. The percentage EGFR levels are relative to un-treated cells.

FIG. 10 shows R0 and R3 AbTAC mediated degradation of PD-L1 is amenable to a variety of different scaffolds. Graph of degradation assays depicting surface levels of PD-L1 as measured by flow cytometry following 24 hr incubation of the indicated AbTAC at different concentrations on T24 R-WT cells. PD-L1 levels are relative to un-treated cells and represent the average of at least three independent biological replicates. Error bars represent one standard deviation of uncertainty.

FIG. 11 shows dummy AbTACs that do not bind a degrader do not degrade PD-L1. Bar graphs of degradation assays showing surface levels of PD-L1 as measured by flow cytometry following 24 hr incubation of the indicated Dummy AbTACs at different concentrations on T24 R-WT cells. PD-L1 levels are relative to un-treated cells. Dummy AbTACs replace the E3 ligase binding arm with a Covid-19 RBD binding arm¹. PD-L1 levels are relative to un-treated cells and represent the average of at least three independent biological replicates. Error bars represent one standard deviation of uncertainty.

FIG. 12 shows Fab Z18 specifically binds ZNRF3 on cells. ZNRF3 Clone 18 specifically binds ZNRF3 on cells. Fab was incubated with HEK 293T cells overexpressing either RNF43 (ECD)-eGFP (RNF43 in which the intracellular domain is replaced with eGFP) or ZNRF3 (ECD)-eGFP (ZNRF3 in which the intracellular domain is replaced with eGFP).

FIGS. 13A-13E show ZNRF3 AbTACs can be utilized to effectively degrade PD-L1 and EGFR. FIGS. 13A-13C are graphs of degradation assays depicting surface levels of PD-L1 as measured by flow cytometry following 24 hr incubation of the indicated AbTAC. 10 nM of AbTAC and T24 Z-WT cells were utilized unless otherwise specified. PD-L1 levels are relative to un-treated cells. FIG. 13A is a graph indicating Z18/Atz mediated PD-L1 degradation is enhanced on T24 cells overexpressing WT ZNRF3 (Z-WT) compared to WT T24 cells (−) FIG. 13B is a graph showing that the affinity of the ZNRF3 binding arm correlates to degradation efficiency. Linear regression analysis was utilized to determine correlation. FIG. 13C is a graph of degradation assays indicating that ZNRF3 mediated degradation of PD-L1 is amenable to a variety of different scaffolds. FIGS. 13G-13H are graphs of degradation assays depicting levels of PD-L1 (FIG. 13D) or EGFR (FIG. 13E) as measured by Western blot following 24 hr incubation of the indicated AbTAC at the indicated concentration on HCC2935 cells. PD-L1 and EGFR levels are relative to un-treated cells. In FIGS. 13A-13E, data are representative of at least three independent biological replicates. PD-L1 and EGFR levels are relative to un-treated cells and represent the average of at least three independent biological replicates. Error bars represent one standard deviation of uncertainty. T tests were utilized to determine significance ****=P<0.0001, ns=not significant.

FIG. 14 shows T24 ZNRF3 overexpression cells. Flow cytometry measurement of the cell surface levels of ZNRF3 on either T24 WT cells or T24 cells overexpressing WT ZNRF3 (T24 Z-WT).

FIGS. 15A-15E show AbTACs do not potentiate Wnt signaling, are not substantially cleared in cell culture, or affected by glycosylation. FIG. 15A shows graphs indicating R0/Atz and Z18/Atz AbTACs do not potentiate canonical WNT signaling on HEK293 STF cells with or without overexpression of PD-L1. Cells were treated with WNT3A and the indicated proteins for 24 hrs prior to determining luciferase activity. FIG. 15B shows BLI measurements indicating that Clones R0, R3, and, Z18 bind a distinct epitope to RSPO2. FIG. 15C shows a serum ELISA demonstrating that AbTACs are not depleted from the media after 24 hr treatment on HCC2935 cells with the indicated concentration of AbTAC. FIG. 15D shows western blot indicating FLAG tagged AbTACs do not accumulate inside cells after 24 hr treatment with 10 nM of the indicated AbTAC on HCC2935 cells. FIG. 15E shows a graph of HCC2935 cells treated with 20 nM of AbTAC with or without glycosylation and measuring levels of PD-L1 degradation after 24 hrs. Results indicate that AbTAC glycosylation status does not markedly affect degradation. T tests were utilized to determine significance, ns=not significant.

### DETAILED DESCRIPTION OF THE DISCLOSURE

The present disclosure generally relates to dual binding agents, which bind to both a membrane-associated ubiquitin E3 ligase and to a target surface protein present on the surface of a target cell. In some embodiments, the present disclosure provides dual binding agents which bind to both a membrane-associated ubiquitin E3 ligase and to a target surface protein present on the surface of a target cell.

In some embodiments, the present disclosure provides exemplary methods to generate constructs comprising IgG fused to an sc-Fv at either the N or C terminus of the IgG light and.or heavy chains. The present disclosure provides methods to test the IgG-scFV fusions. In some embodiments, the present disclosure demonstrates that the dual binding agents of the present disclosure are able to degrade their targets in various clinically relevant cell lines.

The disclosure also provides nucleic acids that encode the dual binding agents, and therapeutic compositions comprising the dual binding agents, and/or nucleic acids encoding the dual binding agents, and cells comprising the nucleic acid. The disclosure also provides methods of treatment using dual binding agents nucleic acids encoding dual binding agents, or therapeutic compositions comprising the dual binding agents and/or nucleic acids encoding the dual binding agents. The disclosure also provides compositions and methods useful for producing such agents, nucleic acids encoding same, host cells genetically modified with the nucleic acids, as well as methods for modulating an activity of a cell and/or for the treatment of various diseases such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a test sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof

The term “treatment” used in reference to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition being treated. Treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention (i.e., reducing the risk of development of clinical symptoms, including causing the clinical symptoms not to develop, e.g., preventing disease progression), and (ii) inhibition (i.e., arresting the development or further development of clinical symptoms, e.g., mitigating or completely inhibiting an active disease).

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

The terms “derivative”, “functional fragment thereof” or “functional variant thereof” refer to a molecule having biological activity in common with the wild-type molecule from which the fragment or derivative was derived. A functional fragment or a functional variant of an antibody is one which retains essentially the same ability to bind to the same epitope as the antibody from which the functional fragment or functional variant was derived. For example, an antibody capable of binding to an epitope of a cell surface receptor may be truncated at the N-terminus and/or C-terminus, and the retention of its epitope binding activity assessed using assays known to those of skill in the art. An antibody derivative may further include constructs based on the general binding properties of antibodies in general, without being directly similar to an existing antibody. For example, one can screen appropriate phage-based libraries for binding to a desired target to obtain binding agents such as nanobodies and scFv agents that are not based on an existing antibody.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### Ubiquitin

Major pathways of protein degradation in eukaryotic cells involve ubiquitination that targets cellular proteins for rapid proteolysis. Ubiquitination is a highly regulated post-translational process that occurs via covalent transfer of ubiquitin to lysine residues of target proteins. The attachment of ubiquitin is mediated by the cooperative action of three classes of enzymes: ubiquitin-activating enzymes (E1), ubiquitin-conjugating enzymes (E2), and ubiquitin-protein ligases (E3). The ubiquitin-activating enzyme E1 activates ubiquitin in an ATP-dependent process to form a thioester linkage between the C-terminal glycine of ubiquitin and a cysteine residue at the E1 active site. The activated ubiquitin is then transferred to a cysteine residue of the ubiquitin-conjugating enzyme E2. The ubiquitin-protein ligase E3 subsequently promotes the transfer of ubiquitin from the E2 enzyme to the lysine residues of protein substrates. Since the human genome encodes two E1 enzymes, about 40 E2 enzymes, and more than 800 E3 ligases, E3 ligases are primarily responsible for conferring substrate specificity in the protein degradation process. Manipulating the substrate specificity of E3 ligases therefore provides a method to redirect the cellular degradation machinery for the targeted proteolysis of proteins of interest.

### Compositions of the Disclosure

As described in greater detail below, the present disclosure provides dual specific binding agents, that are useful for degrading a target surface protein present on the surface of a target cell. Without being bound by any particular theory, these agents are designed to function by binding both a target surface protein and a membrane-associated E3 ligase, such that the target surface protein is ubiquitinated and degraded as a result of binding.

As described in the Examples herein, dual binding agents have been tested and validated in tumor cell lines. Without being bound to any particular theory, it is contemplated that these new agents show similar performance in mouse models and in other mammalian cells, as well as in mammalian subjects, including humans.

### Dual Binding Agents

### Structure

The dual binding agents of the disclosure contain two binding domains: one specific for a membrane-associated E3 ligase, the other specific for a target surface protein. Dual binding agents of the disclosure include agents wherein the E3 ligase binding domain is an IgG scaffold. The target surface protein binding domain is selected from an sc-Fv, single-domain antibodies, nanobodies, Fabs, monospecific Fab2, Fc, minibodies, IgNAR, V-NAR, hcIgG, VHH domains, camelid antibodies, peptibodies, DARPins, and a small molecule. In some embodiments, the target surface protein binding domain is an scFv. The two binding domains of the dual binding agent can be connected through covalent bonds, non-covalent interactions, or a combination thereof.

In some embodiments, the two binding domains of the dual binding agent are attached through a linker. The linker can be a peptide linker, which joins together two binding domains, as described herein. In some embodiments, the length and amino acid composition of the peptide linker sequence can be optimized to vary the orientation, flexibility, and/or proximity of the alteration cassettes relative to one another to achieve a desired activity or property of the dual binding agent.

In some embodiments, a polypeptide linker includes a single-chain polypeptide sequence comprising about 1 to about 30 amino acid residues (e.g., 2, 3, 4, 5, 6, 7, 8, 9, 10, 11, 12, 13, 14, 15, 16, 17, 18, 19, 20, etc. amino acid residues). In some embodiments, a linker sequence includes about 2 to 30, about 3 to 25, about 4 to 20, about 5 to 15, about 6 to 10, about 3 to 15, about 4 to 10, about 5 to 30, about 2 to 5, about 3 to 5, about 4 to 8 amino acid residues.

In some embodiments, the length and amino acid composition of the linker polypeptide sequence can be optimized to vary the orientation, flexibility, and/or proximity of the alteration cassettes relative to one another to achieve a desired activity or property of the encoded polypeptide. In some embodiments, the orientation, flexibility, and/or proximity of the alteration cassettes relative to one another can be varied as a “tuning” tool to achieve a tuning effect that would enhance or reduce the activity of the encoded polypeptide or encoded polypeptide variant. In certain embodiments, the linker contains only glycine and/or serine residues (e.g., glycine-serine linker). Examples of such polypeptide linkers include: Gly, Ser, Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser) n, wherein n is an integer of one or more; and (Ser Gly Gly Gly Gly) n, wherein n is an integer of one or more. In some embodiments, the polypeptide linkers are modified such that the amino acid sequence Gly Ser Gly (GSG) (that occurs at the junction of traditional Gly/Ser linker polypeptide repeats) is not present. In some embodiments, the peptide linker includes the amino acid sequence GGGGSGLNDIFEAQKIEWHEGSSGS (SEQ ID NO: 17) as described in the Examples herein.

The second binding domain can be connected to the first domain in a variety of orientations. In one embodiment, the second binding domain is fused to the C-terminus of the light chain of the IgG scaffold of the first binding domain. In other embodiments, the second binding domain is fused to the C-terminus of the heavy chain of the IgG scaffold of the first binding domain. In some embodiments, the second binding domain is fused to the N-terminus of the heavy chain of the IgG scaffold of the first binding domain.

The dual binding agent can generally take the form of a protein, glycoprotein, lipoprotein, phosphoprotein, and the like. In some embodiments, the target protein binding domain is selected from the group consisting of sc-Fv, single-domain antibodies, nanobodies, Fabs, monospecific Fab2, Fc, minibodies, IgNAR, V-NAR, hcIgG, VHH domains, camelid antibodies, peptibodies, DARPins, and a small molecule. The two binding domains together take the form of a dual binding antibody or derivative thereof. Antibody derivatives need not be derived from a specific wild type antibody. For example, one can employ known techniques such as phage display to generate and select for small proteins having a binding domain similar to an antibody complementarity-determining region (CDR). In some embodiments, the antigen-binding moiety includes an scFv. The binding domain can also be derived from a natural or synthetic ligand or receptor, whether soluble or membrane-bound, that specifically binds to the target surface protein, for example without limitation, PD-1, EGF, and the like.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. In some embodiments, binding affinity is measured by bio-layer interferometry. An antibody that selectively binds an antigen (such as CD19) when it is capable of binding that antigen with high affinity, without significantly binding other antigens.

### Target Surface Proteins

The dual binding agents disclosed herein have a binding affinity for one or more target surface proteins, as well as a membrane-associated E3 ligase. Target surface proteins are selected based on their involvement in immune suppression or the escape of neoplastic cells from immunosurveilance, or their participation in neoplastic cell proliferation or metastasis. Surface proteins that can be targeted according to the methods of the disclosure include proteins such as membrane steroid receptors, EGF receptors, TGF receptors, transferrin receptors, CD19, CD20, CDCP1, and the like. Other suitable target surface proteins include proteins such as PD-L1, PD-L2, CTLA-4, A2AR, B7-H3, B7-H4, BTLA, KIR, LAG3, NKG2D, TIM-3, VISTA, and SIGLEC7, that inhibit attack by immune cells, such as T cells, natural killer cells, macrophages, and the like. In some embodiments, the target surface protein is a protein that is overexpressed by target cells. In some embodiments, the target surface protein is a protein that contributes the target cell's ability to proliferate, metastasize, or evade the immune system. In some embodiments, the target surface protein is an immune checkpoint protein. In some embodiments, the target surface protein is PD-L1, PD-L2, CTLA-4, A2AR, B7-H3, B7-H4, BTLA, KIR, LAG3, NKG2D, TIM-3, VISTA, or SIGLEC7. In some embodiments, the target surface protein is selected from membrane steroid receptors, EGF receptors, TGF receptors, transferrin receptors, CDCP1, CD19, and CD20.

In some embodiments, the target surface protein is a T cell receptor (TCR) polypeptide, a TCR co-stimulatory surface protein, CD4, CD8, or a CAR-T. Bispecific binding agents with this specificity are useful for down-regulating or suppressing T cells and CAR-T cells.

In some embodiments, the dual binding agent is capable of binding a tumor-associated antigen (TAA) or a tumor-specific antigen (TSA). TAAs include a molecule, such as, for example, a protein present on tumor cells and on a sub-population of normal cells, or on many normal cells, but at much lower concentration than on tumor cells. Examples include, without limitation, CEA, AFP, HER2, CTAG1B and MAGEA1. In contrast, TSAs generally include a molecule, such as a protein present on tumor cells but not expressed on normal cells. Examples include, without limitation, oncoviral antigens and mutated proteins (also known as neoantigens).

In some cases, the target surface protein binding domain is specific for an epitope present in an antigen that is expressed by a malignant neoplastic cell, e.g., a tumor-associated antigen or a tumor-specific antigen. The tumor-associated or tumor-specific antigen can be an antigen associated with, for example, a breast cancer cell, a B cell lymphoma, a pancreatic cancer, a Hodgkin's lymphoma cell, an ovarian cancer cell, a prostate cancer cell, a mesothelioma, a lung cancer cell, a non-Hodgkin's B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a bladder cancer cell, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

In some embodiments, the dual binding agent of the present disclosure can induce degradation of the of the target protein. In some embodiments, the degradation of the target protein is induced with a Dmax of at least 20%, 25%, 30%, 35%, 40%, 45%, 50%, 55%, 60, 65%, 70%, 75%, 80%, 85%, 90%, 95%, or 100%.

In some embodiments, degradation of the target protein reduces the ability of the target cell to proliferate

### E3 Ligases

The dual binding agent of the disclosure also binds a membrane-associated E3 ligase. E3 ligases useful in the disclosure include those ligases that are found in association with the target cell plasma membrane (cell membrane). These membrane-associated E3 ligases include, for example, RNF43, ZNRF3, RNF133, RNF148, GRAIL (RNF128), RNF149, Goliath (RNF130), RNF150, RNF122, ZNRF4, RNF13, RNF167, RNF121, RNF175, DCST1, March6, Kf-1 (RNF103), RNF182, RNF145, TRC8 (RNF139), HRD1 (SYVN1), RNFT1, MAPL (MUL1), RNF152, RNF26, RINES (RNF180), MARCHF3, MARCHF2, MARCHF8, MARCH1 (MARCHF1), March11, MARCHF9, March4, RNF186, RNF170, RNF185, RMA1 (RNF5), TRIM59 (RNF104), TRIM13, MARCHF5 (MARCH5), RNF197 (CGRF1), RNF183, RNF217, RNF144B, RNF144A, RNF19B, and RNF19A, and the like. GRAIL (RNF128) is characteristically expressed in T cells; thus the activity of a dual binding agent that binds to RNF128 can be limited to T cells and any other cells that express RNF128.

### Exemplary Constructs

In some embodiments, the dual binding agent of the present disclosure comprises a first binding domain to an E3 ligase that is an IgG scaffold and a second domain for a target surface protein that is an scFv as provided herein. In some embodiments, the first binding domain to an E3 ligase binds to an extracellular protein attached to an E3 ligase or a transmembrane protein that interacts with an E3 ligase.

In certain embodiments, the binding domain an E3 ligase comprises an IgG scaffold comprising a heavy chain and Fc domain amino acid sequence as set forth in SEQ ID NO: 1 below. The Fc region is underlined.

EISEVQLVESGGGLVQPGGSLRLSCAASGFNIYYYSMHWVRQAPGKGLE
WVASISPYYSYTSYADSVKGRFTISADTSKNTAYLQMNSLRAEDTAVYY
CARYGYYGWDYHRYSAFDYWGQGTLVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGK

In some embodiments, the binding domain an E3 ligase comprises an IgG scaffold comprising a heavy chain and Fc domain amino acid sequence as set forth in SEQ ID NO: 2 below. The Fc region is underlined.

In some embodiments, the binding domain an E3 ligase comprises an IgG scaffold comprising a heavy chain and Fc domain amino acid sequence as set forth in SEQ ID NO: 2 below. The Fc region is underlined.

EISEVQLVESGGGLVQPGGSLRLSCAASGFNIYYYSIHWVRQAPGKGLE
WVASIYSSSGYTSYADSVKGRFTISADTSKNTAYLQMNSLRAEDTAVYY
CARYPYWYFDGFDYWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

In some embodiments, the binding domain an E3 ligase comprises an IgG scaffold comprising an amino acid sequence as set forth in SEQ ID NO: 3 below.

EISEVQLVESGGGLVQPGGSLRLSCAASGFNLYYSYIHWVRQAPGKGLE
WVASIYPSYGSTYYADSVKGRFTISRDNSKNTLYLQMNSLRAEDTAVYY
CARGYAIDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

In some embodiments, the second binding domain comprises the amino acid sequence set forth in SEQ ID NO: 4 below.

DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKGGGGSGGGGSGGGGSEVQLVESGGGLVQPGGSLRLSCAAS
GFTFSDSWIHWVRQAPGKGLEWVAWISPYGGSTYYADSVKGRFTISADT
SKNTAYLQMNSLRAEDTAVYYCARRHWPGGFDYWGQGTLVTVSS

In some embodiments, the first binding domain comprises the heavy chain-Fc region sequence set forth in SEQ ID NOs.: 1 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

In some embodiments, the first binding domain comprises the heavy chain-Fc region sequence set forth in SEQ ID NOs.: 2 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

In some embodiments, the first binding domain comprises the heavy chain-Fc region sequence set forth in SEQ ID NOs.: 3 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

Exemplary sequences in accordance with these embodiments include SEQ ID Nos: 5-10. In some embodiments, the dual binding agents of the disclosure include an amino acid sequence having at least 80%, at least 85%, at least 90%, at least 95%, at least 96%, at least 97%, at least 98%, at least 99%, or 100% sequence identity to an amino acid sequence of SEQ ID NOs: 5-10.

In certain embodiments, the binding domain an E3 ligase comprises an IgG scaffold comprising a light chain domain amino acid sequence as set forth in SEQ ID NO: 11 below.

DIQMTQSPSSLSASVGDRVTITCRASQSVGSALAWYQQKPGKAPKLLIY
SASSLYSGVPSRFSGSRSGTDFTLTISSLQPEDFATYYCQQAYPITFGQ
GTKVEIKRTVAAPSVFIFPPSDSQLKSGTASVVCLLNNFYPREAKVQWK
VDNALQSGNSQESVTEQDSKDSTYSLSSTLTLSKADYEKHKVYACEVTH
QGLSSPVTKSFNRGEC

In certain embodiments, the binding domain an E3 ligase comprises an IgG scaffold comprising a light chain domain amino acid sequence as set forth in SEQ ID NO: 12 below.

DIQMTQSPSSLSASVGDRVTITCRASQSVGSALAWYQQKPGKAPKLLIY
SASSLYSGVPSRFSGSRSGTDFTLTISSLQPEDFATYYCQQGYSDLITF
GQGTKVEIKRTVAAPSVFIFPPSDSQLKSGTASVVCLLNNFYPREAKVQ
WKVDNALQSGNSQESVTEQDSKDSTYSLRSTLTLSKADYEKHKVYACEV
THQGLSSPVTKSFNRGEC

In certain embodiments, the binding domain an E3 ligase comprises an IgG scaffold comprising a light chain domain amino acid sequence as set forth in SEQ ID NO: 13 below.

DIQMTQSPSSLSASVGDRVTITCRASQSVGSALAWYQQKPGKAPKLLIY
SASSLYSGVPSRFSGSRSGTDFTLTISSLQPEDFATYYCQQSYYPITFG
QGTKVEIKRTVAAPSVFIFPPSDSQLKSGTASVVCLLNNFYPREAKVQW
KVDNALQSGNSQESVTEQDSKDSTYSLSSTLTLSKADYEKHKVYACEVT
HQGLSSPVTKSFNRGEC

In some embodiments, the first binding domain comprises the light chain region sequence set forth in SEQ ID NO.: 11 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

In some embodiments, the first binding domain comprises the heavy chain-Fc region sequence set forth in SEQ ID NO.: 12 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

In some embodiments, the first binding domain comprises the heavy chain-Fc region sequence set forth in SEQ ID NO.: 13 or a variant thereof comprising 1, 2, 3, or 4 conservative amino acid substitutions, and the second binding domain comprises SEQ ID NO: 4 or a variant thereof comprising 1, 2, 3, or 4 conservative amino acid substitutions. In some embodiments, the first binding domain and the second binding domain are linked by a linker. In some embodiments, the first binding domain and second binding domain sequence comprise sequences that are about 70%, 75%, 80%, 85%, 90%, 95%, 99% identical to the sequences provided herein.

Exemplary sequences in accordance with these embodiments include SEQ ID Nos: 14-16. In some embodiments, the dual binding agents of the disclosure include an amino acid sequence having at least 80%, at least 85%, at least 90%, at least 95%, at least 96%, at least 97%, at least 98%, at least 99%, or 100% sequence identity to an amino acid sequence of SEQ ID NOs: 14-16.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the binding agents of the present disclosure do not abrogate the binding of the binding agent containing the amino acid sequence, to the antigen(s), i.e., the E3 ligase and/or the target surface protein to which the binding agent binds. Methods of identifying nucleotide and amino acid conservative substitutions which do not eliminate antigen binding are well-known in the art.

As shown in the Examples provided herein, some of the dual bnding agents exhibiting a surprising increase in Dmax (up to 97% and 99%) when compared to previously generated bispecific constructs. This activity translates to a greater extent of target protein degradation, which would not be expected with a much larger construct such as the dual binding agents described herein. By way of example, the dual binding agents of the present disclosure are in a traditional IgG format requiring 2 copies of all heavy and light chains. This is in contrast to the much less bulky bispecific agent that have been described previously.

### Synthesis

Dual binding agents are synthesized using the techniques of recombinant DNA and protein expression. For example, for the synthesis of DNA encoding a dual IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Nucleic Acid Molecules

In one aspect, some embodiments disclosed herein relate to nucleic acid molecules comprising nucleotide sequences encoding the dual binding agents of the disclosure, including expression cassettes, and expression vectors containing these nucleic acid molecules operably linked to heterologous nucleic acid sequences such as, for example, regulatory sequences which direct in vivo expression of the protein in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a dual binding agent operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. Vectors generally comprise a recombinant polynucleotide construct designed for transfer between host cells, that may be used for the purpose of transformation, i.e., the introduction of heterologous DNA into a host cell. As such, in some embodiments, the vector can be a replicon, such as a plasmid, phage, or cosmid, into which another DNA segment may be inserted so as to bring about the replication of the inserted segment. Expression vectors further include a promoter operably linked to the recombinant polynucleotide, such that the recombinant polynucleotide is expressed in appropriate cells, under appropriate conditions. In some embodiments, the expression vector is an integrating vector, which can integrate into host nucleic acids.

In some embodiments, the expression vector is a viral vector, which further includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Retroviral vectors contain structural and functional genetic elements, or portions thereof, that are primarily derived from a retrovirus. Lentiviral vectors are viral vectors or plasmids containing structural and functional genetic elements, or portions thereof, including LTRs that are primarily derived from a lentivirus.

The nucleic acid sequences can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the proteins disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the proteins disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any dual binding agent disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a dual binding agent as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a dual binding agent) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

### Recombinant Cells and Cell Cultures

The nucleic acid of the present disclosure can be introduced into a host cell, such as a human B lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses. An embodiment is an AAV vector encoding the engineered transmembrane protein of the disclosure.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells are genetically engineered (e.g., transduced, transformed, or transfected) with, for example, a vector comprising a nucleic acid sequence encoding an engineered transmembrane protein as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the dual binding agents, nucleic acids, and recombinant cells of the disclosure can be incorporated into compositions, including pharmaceutical compositions. Such compositions typically include the dual binding agents, nucleic acids, and/or recombinant cells, and a pharmaceutically acceptable excipient, e.g., a carrier.

Dual binding agents of the disclosure can be administered using formulations used for administering antibodies and antibody-based therapeutics, or formulations based thereon. Nucleic acids of the disclosure are administered using formulations used for administering oligonucleotides, antisense RNA agents, and/or gene therapies such as CRISPR/Cas9 based therapeutics.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the dual binding agents of the disclosure are administered by transfection or infection with nucleic acids encoding them, using methods known in the art, including but not limited to the methods described in McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

### Methods of the Disclosure

### Administration of Dual Binding Agents

Administration of any one or more of the therapeutic compositions described herein, e.g., dual binding agents, nucleic acids, recombinant cells, and pharmaceutical compositions, can be used to treat individuals having a neoplastic disease, such as cancers. In some embodiments, the dual binding agents, recombinant cells, and pharmaceutical compositions are incorporated into therapeutic compositions for use in methods down-regulating or inactivating T cells, such as CAR-T cells.

Accordingly, in one aspect, provided herein are methods for inhibiting an activity of a target cell in an individual, the methods comprising the step of administering to the individual a first therapy including one or more of the dual binding agents, nucleic acids, recombinant cells, and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual by expression of dual binding agents. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a bladder cancer cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

Dual binding agents of the disclosure are typically administered in solution or suspension formulation by injection or infusion. In an embodiment, a dual binding agent is administered by injection directly into a tumor mass. In another embodiment, a dual binding agent is administered by systemic infusion.

Some dual binding agents of the disclosure are effective at a concentration of 10 nM. Other dual binding agents may be most effective at a higher or lower concentration, depending on the binding affinity for each of the ligands, and the degree of expression of each of the ligands. The range of effective concentrations, however, can be determined by one of ordinary skill in the art, using the disclosure and the experimental protocols provided herein. Similarly, using the effective concentration one can determine the effective dose or range of dosages required for administration.

Depending on the disease or disorder to be treated, the severity and extent of the disease, the subject's health, and the co-administration of other therapies, repeated doses may be administered. Alternatively, a continuous administration may be required. It is expected, however, that the dual binding agent will remain in proximity to the cell so that each molecule of dual binding agent can ubiquitinate and degrade multiple molecules of target surface protein. Thus, the dual binding agents of the disclosure may require lower doses, or less frequent administration, than therapies based on antibody competitive binding.

### Administration of Recombinant Cells to an Individual

In some embodiments, the methods involve administering the recombinant cells to an individual who is in need of such method. This administering step can be accomplished using any method of implantation known in the art. For example, the recombinant cells can be injected directly into the individual's bloodstream by intravenous infusion or otherwise administered to the individual.

The terms “administering”, “introducing”, and “transplanting” are used interchangeably herein to refer to methods of delivering recombinant cells expressing the dual binding agents provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the life time of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2× 10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2× 10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells a dual binding agent provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. Injection modes include, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; (2) relieving the disease, e.g., causing regression of symptoms; or (3) preventing or reducing the likelihood of the development of symptoms.

In some embodiments, the individual is a mammal. In some embodiments, the mammal is human. In some embodiments, the individual has or is suspected of having a disease associated with cell signaling mediated by a cell surface protein. In some embodiments, the disease is a cancer or a chronic infection.

### Systems and Kits

Also provided herein are systems and kits including the dual binding agents, recombinant nucleic acids, recombinant cells, or pharmaceutical compositions provided and described herein as well as written instructions for making and using the same. For example, provided herein, in some embodiments, are systems and/or kits that include one or more of: a dual binding agent as described herein, a recombinant nucleic acid as described herein, a recombinant cell as described herein, or a pharmaceutical composition as described herein. In some embodiments, the systems and/or kits of the disclosure further include one or more syringes (including pre-filled syringes) and/or catheters used to administer one any of the provided dual binding agents, engineered transmembrane proteins, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the dual binding agents.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Materials and Methods for Examples

Cell culture: HEK 293T, T24, and HCC2935 cell lines were grown and maintained at 37° C. and 5% C02. HEK293T cells were grown in DMEM supplemented with 10% fetal bovine serum (FBS) and 1% penicillin/streptomycin (P/S). T24 cells were grown in McCoy's 5a supplemented with 10% FBS and 1% P/S. HCC2935 cells were grown in RPMI supplemented with 10% FBS and 1% P/S. T24 and HEK 293T WT cells were obtained from the UCSF Cell Culture Facility. HEK 293 Super Top Flash (STF) WNT reporter cells (HEK 293 Stf) and HCC2935 cells were obtained from the American Type Culture Collection (ATCC).

Bio-Layer Interferometry (BLI): BLI data was measured using an Octet RED384 (ForteBio) instrument. RNF43 (Extracellular domain)-Fc or ZNRF3 (Extracellular domain)-Fc fusions were immobilized on a streptavidin biosensor and loaded until 1.0 nm signal was achieved. After blocking with 10 μM Biotin, purified Fabs at the indicated concentrations in Phosphate buffered saline containing 0.1% Tween and 0.2% Bovine serum albumin (PBS-T 0.2% BSA) were used as the analytes. Data were analyzed using the ForteBio Octet analysis software and kinetic parameters for each Fab were determined using either a 1:1 monovalent (For RNF43 R0 clones and ZNRF3 Z18 clones) or 1:2 heterobifunctional binding model (For RNF43 R3 and R6 clones). Epitope binning experiments for RNF43 clones R0, R3, and R6 were performed by first incubating RNF43-Fc bound tips with 50 nM of Fab1 and then with both 50 nM Fab1 and 25 nM of Fab2. Epitope binning experiments for binding with RNF43 clones R0 and R3 as well as Znrf3 Clone Z18 and R-spondin 2 were performed by first incubating RNF43-Fc or Znrf3 bound tips with 50 nM of the indicated Fab and then 100 nM of R-spondin2 and 50 nM of the indicated Fab.

Generation of Overexpression cell lines: T24 R-WT, T24 R-MUT, T24 Z-WT, HEK 293T R-MUT, and HEK 293T Z-MUT overexpression cells were made using a lentiviral transduction system: R-WT=N-terminally Myc Tagged RNF43, R-MUT=N-terminally Myc Tagged RNF43 in which the C-terminal intracellular domain has been replaced with eGFP, Z-WT=N-terminally Myc Tagged ZNRF3, Z-MUT=N-terminally Myc Tagged ZNRF3 in which the C-terminal intracellular domain has been replaced with eGFP. The indicated protein sequences were cloned into a pCDH-EF1-FHC (Addgene plasmid #64874) vector and then each was transfected along with standard packaging vectors into HEK 293T cells to generate lentivirus. Media containing virus was collected 72 hrs after transfection and filtered using a 0.45 μM filter. The filtered lentivirus containing media was then used to transduce either T24 or HEK293T cells and stably transduced cells were selected for with Puromycin (0.8 μg/ml). Successful transduction and expression of each protein was confirmed using flow cytometry.

Expression and Purification of Fabs: Fabs were expressed and purified using an optimized autoinduction protocol that has been previously described2. In brief, C43 (DE3) Pro+E. coli containing expression plasmids for the indicated Fabs were grown in TB autodinduction media at 37° C. for 6 hrs and then switched to 30° C. for roughly 18 hrs. Fabs were purified by Protein A affinity chromatography and buffer exchanged into PBS. Purity was assessed by SDS/PAGE.

Flow cytometry: Cells were washed with room temperature PBS, and then lifted using Versene. Cells were then added to wells of a 96 well dish and pelleted by centrifugation (500 g, 5 min, 4° C.). Cell pellets were washed 1× with 200 ul cold PBS containing 3% BSA and then pelleted again by centrifugation. Cells were then resuspended and incubated with 200 ul of cold PBS containing 3% BSA and the indicated primary antibody at a concentration of 10 mg/ml for 20 minutes on ice. For all PD-L1 degradation assays, cells were incubated with 200 ul of cold PBS containing 3% BSA and a final concentration of 50 nM Atezolizumab for 20 minutes. Following incubation with primary antibody, cells were washed three times with 200 ul of cold PBS containing 3% BSA and then resuspended and incubated with 200 ul of cold PBS containing 3% BSA and the indicated secondary antibody at a dilution of 1:1000. For all PD-L1 degradation assays, cells were incubated with 200 ul of cold PBS containing 3% BSA and a 1:500 dilution of (D8T4X)-647 (Cell Signaling) or a Rabbit IgG-647 (Cell signaling) isotype control. Following 30 minutes of incubation on ice, cells were washed 3× with 200 ul of cold PBS containing 3% BSA. Flow cytometry was performed on a CytoFLEX cytometer (Beckman Coulter) and gating was performed on single cells before acquisition of cells. Analysis was performed using the FLowJo software package. Surface PD-L1 levels were determined by measuring median APC signal.

Western blotting: Cells were washed with room temperature PBS, lifted using Versene and pelleted by centrifugation (500 g, 5 min, 4° C.). Cells were then lysed with RIPA lysis buffer containing complete mini protease inhibitor (Sigma) on ice for 20 minutes. Lysates were pelleted (17000 g, 10 min, 4° C.) and the supernatant was removed and mixed with 4× NuPAGE LDS sample buffer (Invitrogen) and 1% BME. Equal amounts of lysates were loaded onto a 4-12% Bis-Tris gel and ran at 200V for 40 minutes. The gel was then transferred to a polyvinylidene difluoride (PVDF) membrane using an iBLot2 and standard manufacturer protocol (Thermo). The membrane was then blocked in Odyssey Blocking Buffer (TBS) (LICOR) for 45 minutes at room temperature with gentle shaking. The membranes were then incubated overnight with primary antibodies for anti-β-Actin (8H10D10: Cell Signaling), and either anti-PD-L1 (E1L3N: Cell Signaling) or anti-EGFR (D38B1: Cell Signaling). Following overnight incubation, membranes were washed three times with TBS+0.1% Tween (TBS-T) and then incubated with secondary antibodies for 1 hr at room temperature: 680RD Goat anti-mouse IgG and 800RD Goat anti-Rabbit IgG (LI-COR Biosciences). Membranes were washed again 3× with TBS-T and then analyzed using an OdysseyCLxImager (LI-COR Biosciences). Band intensities were quantified using Image Studio Software (LI-COR Biosciences).

Fab-Phage display selections: Phage display was performed as previously described7. In brief, selections with Fab-phage Library E and Library UCSF were performed using biotinylated RNF43-Fc or ZNRF3-Fc fusions as the positive antigens and Biotinylated Fc for the negative selections. A ‘Catch and release’ strategy was utilized with streptavidin-coated magnetic beads (Promega) and TEV protease. Four rounds of selections were used with each successive round using a decreased concentration of Antigen (1000 nM, 100 nM, 50 nM, 10 nM) to selective for higher affinity binders. 96 clones were then chosen to be analyzed using a Fab-phage ELISA as previously described⁷. Clones that looked promising via ELISA were then sequenced to determine the identity of the CDRs. Unique clones were cloned into a Fab Expression vector and expressed and purified as Fabs and analyzed by BLI.

Degradation experiments: For all degradation assays using T24 cells, the indicated T24 cells were plated into wells of a 12 well dish at a density of between 10,000 and 15,000 cells per well and allowed to grow for roughly 72 hrs. Cells were then treated with 10 nM (unless otherwise indicated) of AbTAC for 24 hrs. For all PD-L1 degradation assays on T24 cells, cells were analyzed using the flow cytometry workflow, described in detail in the Supplementary Materials and Methods section. Background APC-A signal from cells incubated with isotype control was subtracted for each of the samples and the relative surface levels of PD-L1 were then determined by dividing the signal of each treated sample by the signal of the untreated sample. For all EGFR degradation assays using T24 cells, the previously described Western blotting protocol was used. We determined the relative total levels of EGFR, by calculating the ratio of EGFR/β-Actin for each individual sample and dividing this by the ratio of EGFR/β-Actin for the untreated sample. For all degradation assays using HCC2935 cells, cells were plated into wells of 6 well dish. When cells were at roughly at 70% confluency, they were treated with AbTAC for 24 hrs. Cells were then harvested and analyzed using the described Western blot workflow to determine relative levels of PD-L1 and EGFR. To determine DC50 values, data points were plotted using GraphPad Prism (version 9.1), and curves to determine DC50 were generated by using nonlinear regression with Sigmoidal 4PL parameters.

Wnt Activation assay: HEK 293T cells that have been stably transduced with Firefly Luciferase Reporter under the control of seven LEF/TCF binding sites (HEK 293 STF) were purchased from ATCC. These cells were further transiently transfected to express PD-L1 under a CMV promoter. 36 hours after transfection, cells were stimulated with 20% WNT3a conditioned media (gift from the Mattis Lab at UCSF) supplemented with 25 nM RSPO2 (R&D Systems), R0/Atz (10 nM or 100 nM), Z18/Atz (10 nM or 100 nM), or a 1:1 mixture of R0/Atz and Z18/Atz (10 nM or 100 nM). Cells were cultured in the presence of reagents for another 24 hours before the addition of ONE-Glo Luciferase reagent (Promega). Cells were incubated in the dark for 15 minutes before being transferred to a white 96-well plate and imaged using a Tecan Infinite M200 Pro plate reader and analyzed by GraphPad Prism 7.

Serum ELISA assay: HCC2935 cells were dosed for 24 hours with either described AbTAC. Prior to lifting cells, PBS controls were treated with corresponding AbTAC for 10 minutes. Media was harvested and placed on ice. Cells were washed with cold PBS, lifted with Versene solution, and pelleted by centrifugation (500 g, 5 min 4° C.) and samples prepared for Western blotting to detect a flag-tag using mouse-anti-FLAG (Sigma Aldrich: Clone M2, 1:1000). 24 hours prior to media harvesting each well in a 384-well Fischer Maxisorp plate was coated with 0.5 μg/mL NeutrAvidin in PBS, and the plate incubated overnight at 4° C. Coating solution was removed, and plate blocked with PBS+0.05% Tween-20+0.2% BSA (blocking buffer) for 1 hour at RT. 40 nM biotinylated protein A or blocking buffer was added and incubated for 20 minutes. Antigen solution was removed, and 1 μM biotin in blocking buffer was added and incubated for 10 minutes. Plate was washed three times with PBS+0.05% Tween-20. Harvested media was added and incubated for 20 minutes. Media was removed and plate washed three time with PBS+0.05% Tween-20. Protein-L HRP conjugated antibody (Thermo Scientific: #32420) diluted 1:5000 in blocking buffer was added and incubated for 30 minutes at RT. Antibody solution was removed and plate washed three time with PBS+0.05% Tween-20. HRP TMB substrate was added and incubated until signal appeared. Reaction was quenched with 1 M phosphoric acid and plates analyzed at OD450 nm on a SpectraMax plate reader

### Example 2

### Levels of E3 Ligase and E3-Ligase Domain Affects Degradation Efficiency

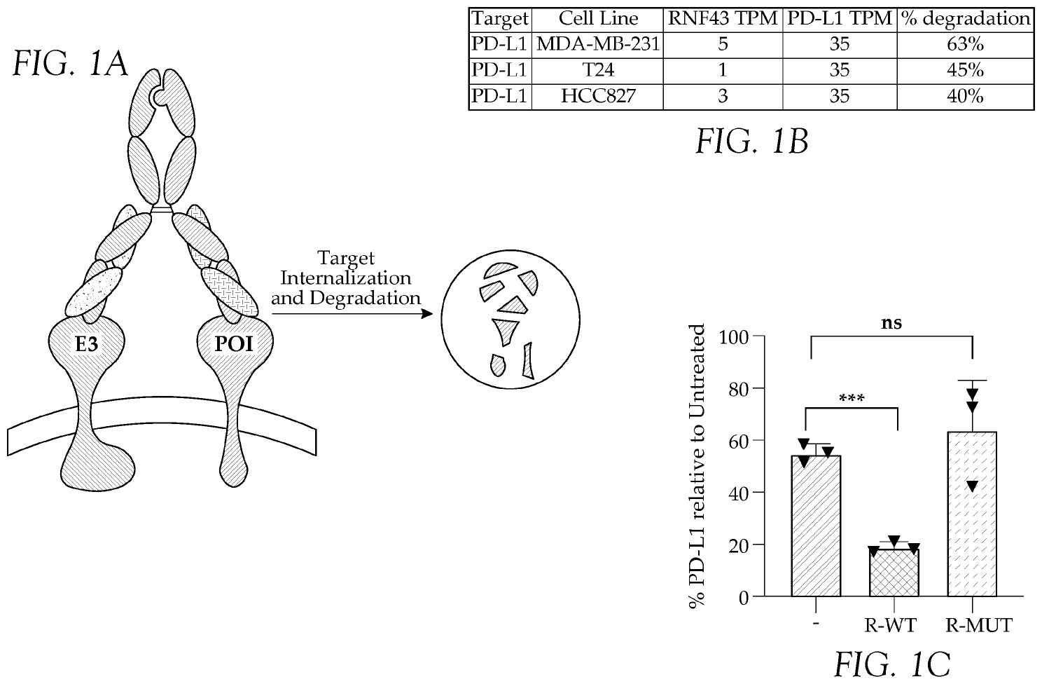

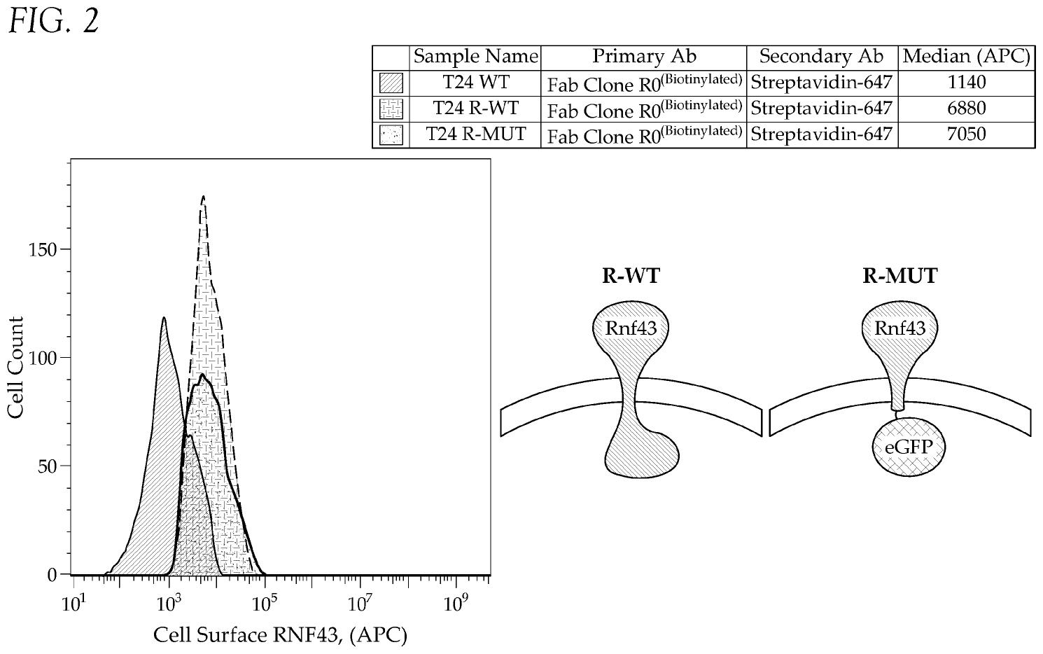

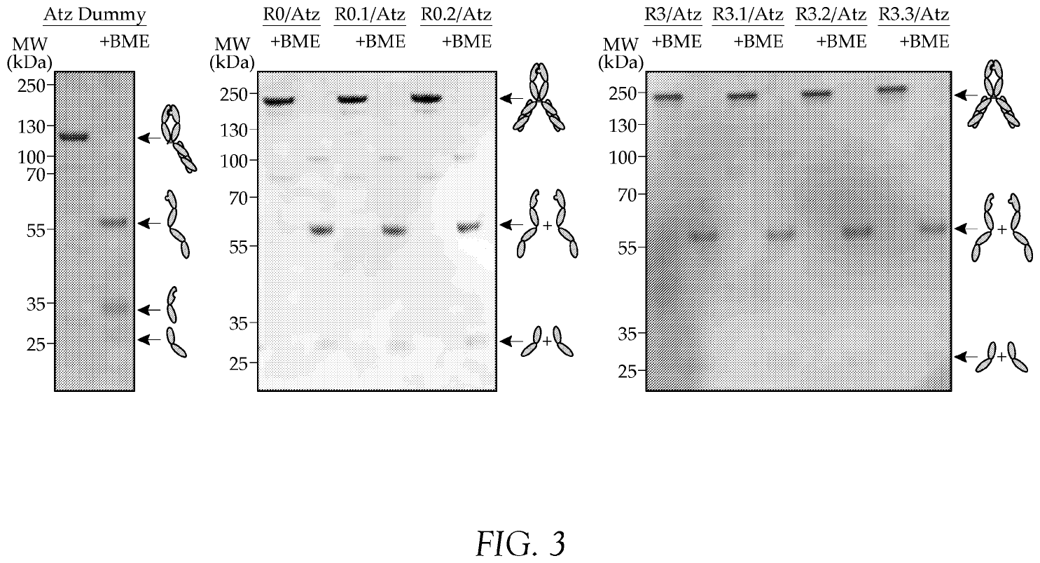

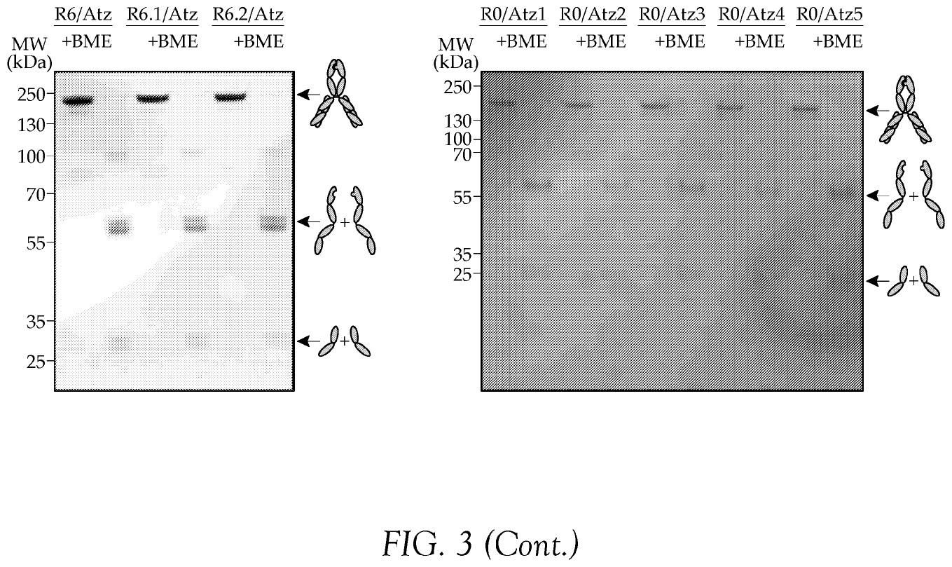

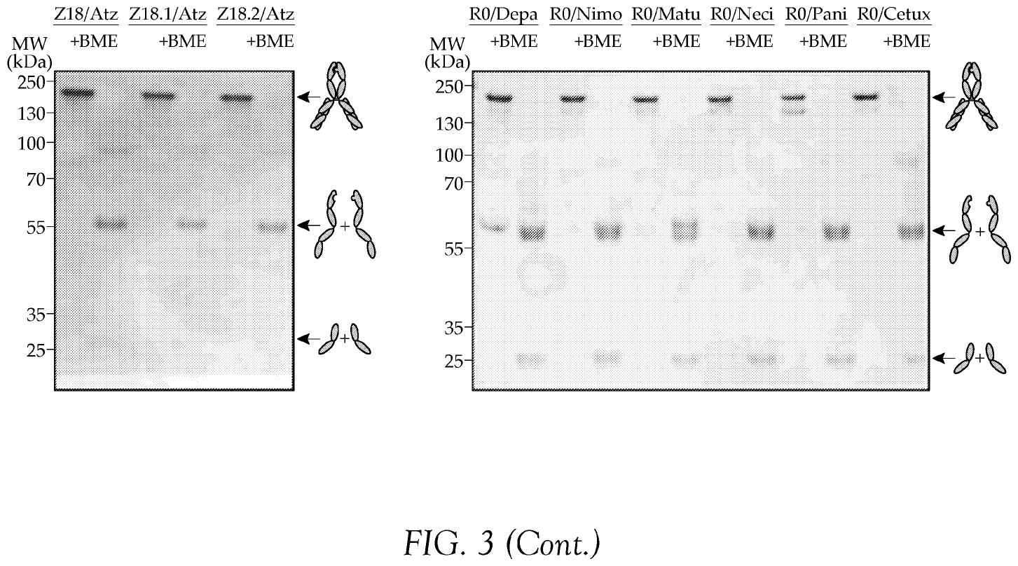

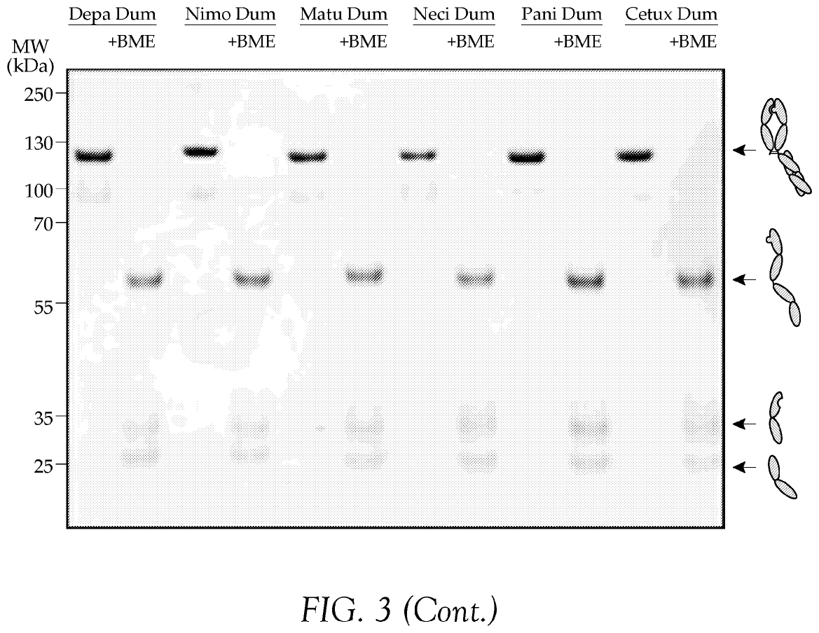

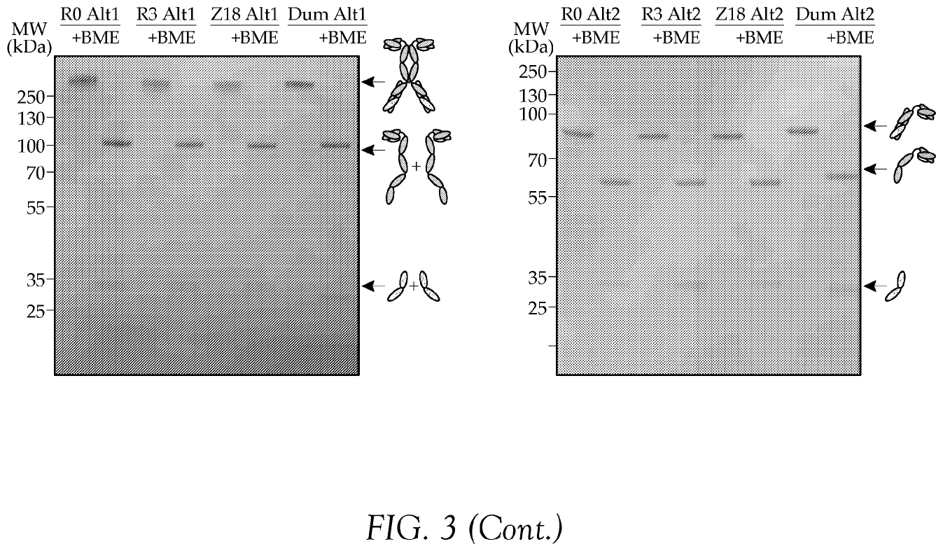

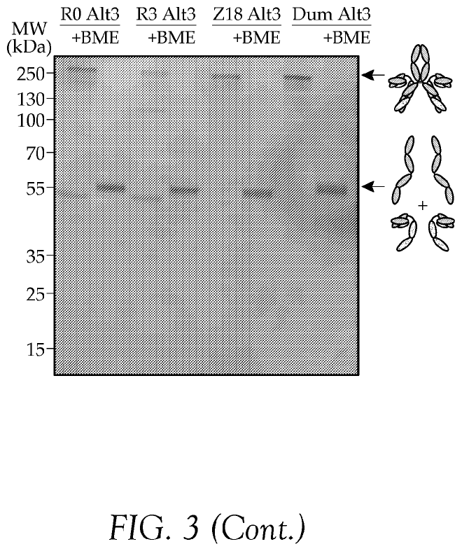

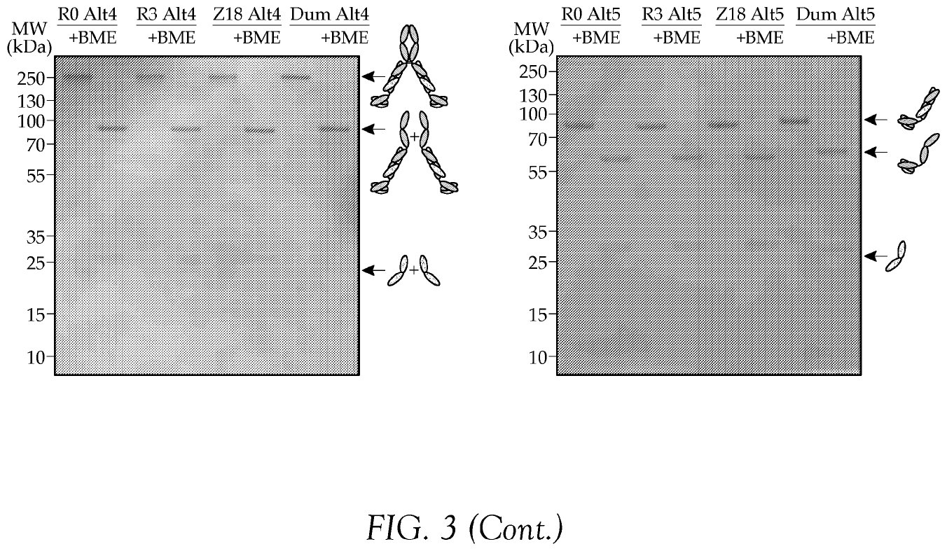

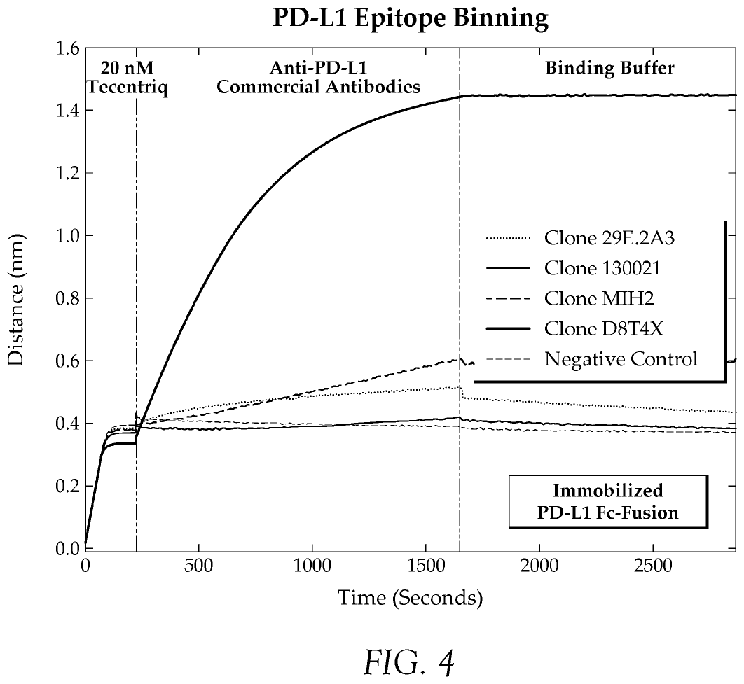

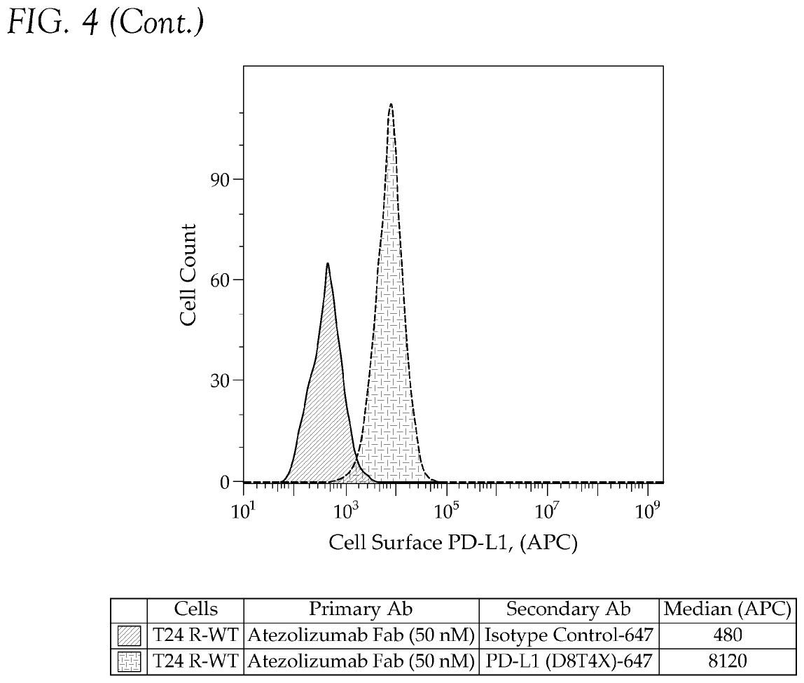

Our previous work in developing AbTACs utilized a standard bispecific IgG format to bring a cell surface E3 ligase (RNF43) in proximity of the immune-checkpoint protein programmed death-ligand 1 (PD-L1) to mediate its degradation through the lysosomal degradation pathway (FIG. 1A). Although we were able to successfully degrade PD-L1 on three different cell lines, the maximal degradation we were able to achieve was ˜60%, suggesting room for optimization⁷ (FIG. 1B). We hypothesized that low cell surface levels of RNF43 relative to PD-L1 might be preventing more efficient POI degradation. While we believe that the data indicate that AbTACs may function catalytically, a large excess of POI relative to E3 ligase may still overwhelm the E3 ligase degradation machinery. To this end, we found that RNA transcript levels were 7-10 fold higher for the target compared to the E3 ligase. 9 (FIG. 1B). Because RNA transcript levels do not always correlate directly with proteins levels, we decided to more directly test if degradation would improve with increased surface levels of RNF43. Therefore, we generated T24 cell lines overexpressing either WT RNF43 (T24 R-WT) or an RNF43 mutant that replaces the intracellular E3 ligase containing domain with an inert fluorescent protein, eGFP (T24 R-MUT) (FIG. 2). We generated a bispecific IgG AbTAC, R0/Atz, as before⁷ (FIG. 3) using the standard Knob-into-Hole engineering strategy¹⁰. The RNF43 binding arm is an antigen binding fragment (Fab) previously isolated from our in-house Fab phage library (clone R0); the PD-L1 binding arm is from the clinically approved monoclonal antibody, atezolizumab (Atz). To measure surface levels of PD-L1, we utilized a flow-based system with a commercially available fluorescent antibody that binds a separate epitope from atezolizumab (FIG. 4). Gratifyingly, when we performed a degradation assay with R0/Atz on T24 R-WT cells and measured surface levels of PD-L1 after 24 hrs, we found that PD-L1 degradation increased to 80%. In contrast, overexpression of the mutant RNF43 did not increase degradation over the control (FIG. 1C). These data indicate that while degradation can proceed even when RNF43 is less abundant than the target, it proceeds more efficiently on cells containing higher levels of fully functional RNF43. As such, RNF43 expression levels should be a consideration when choosing cell lines to target.

### Example 3

### Binding Epitope on the Degrader Matters, and Only Requires Modest Affinity

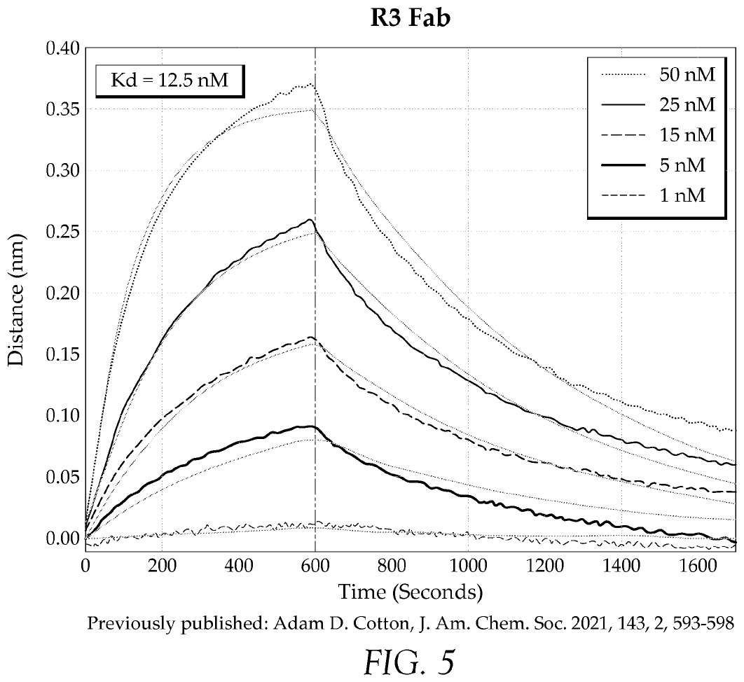

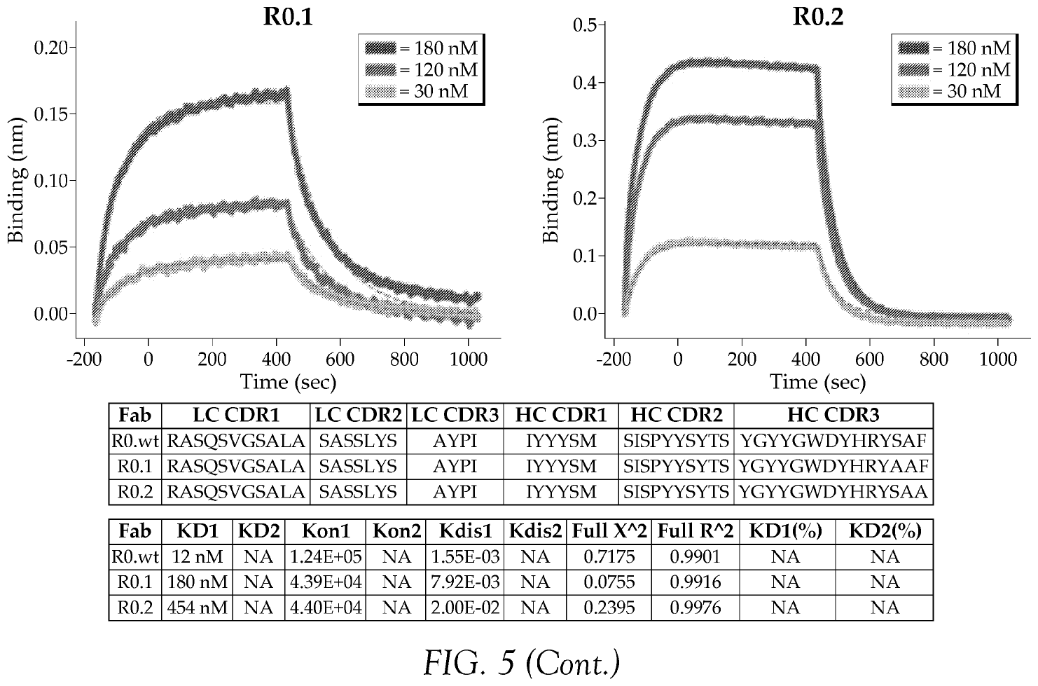

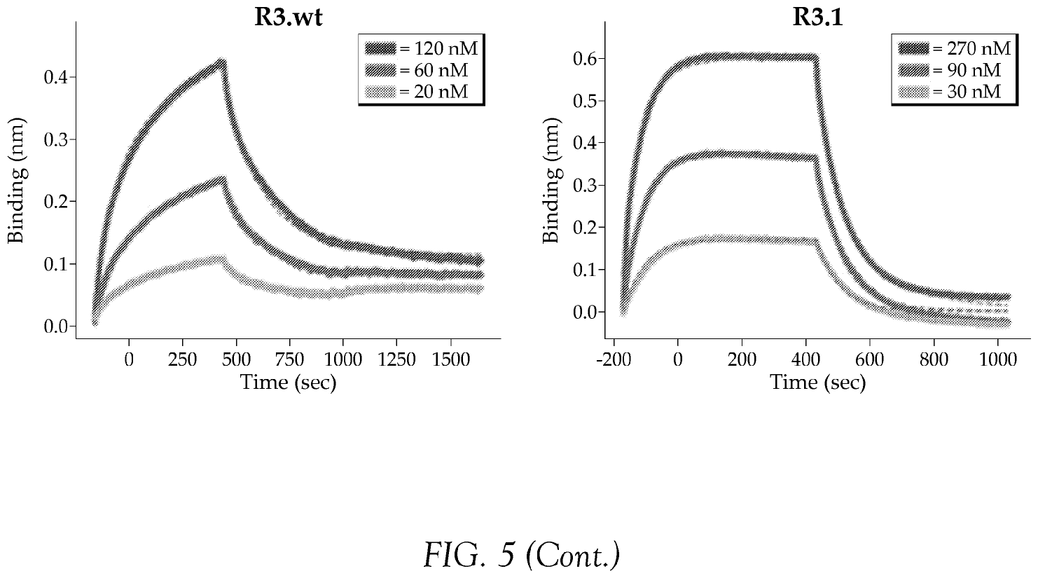

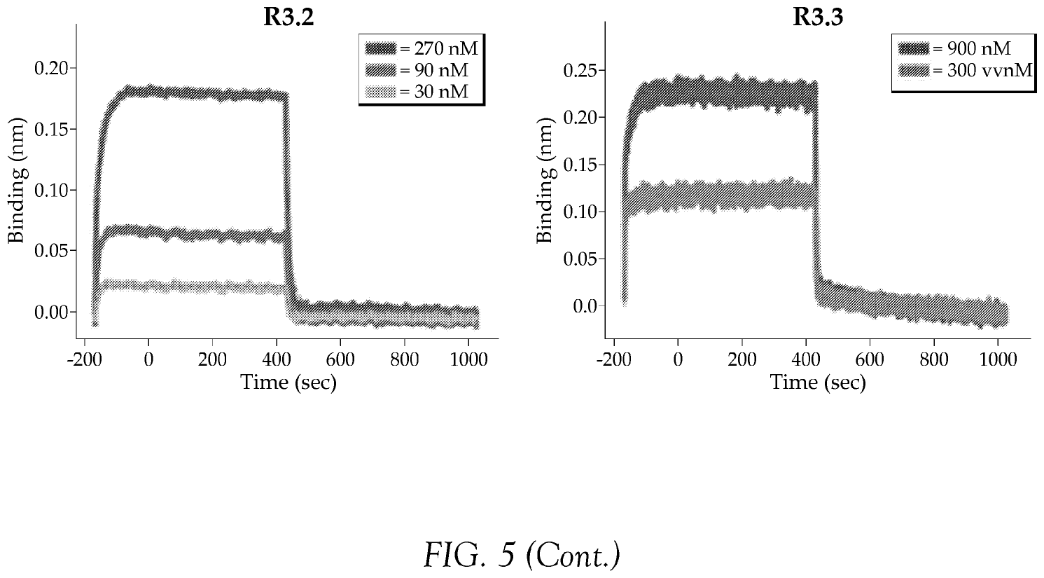

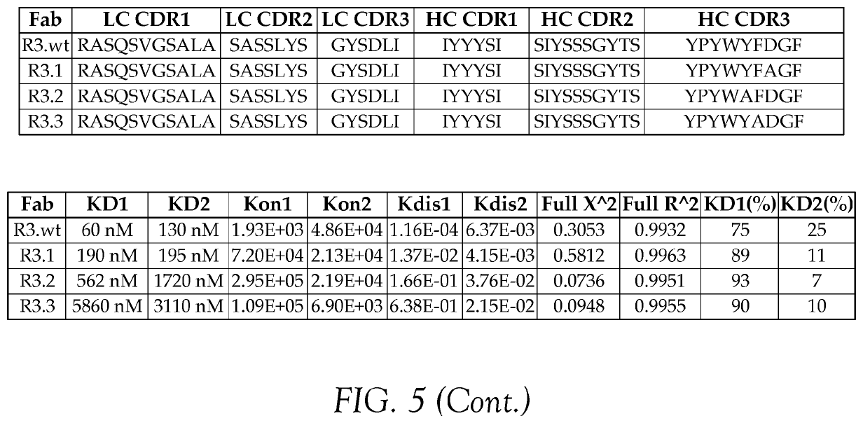

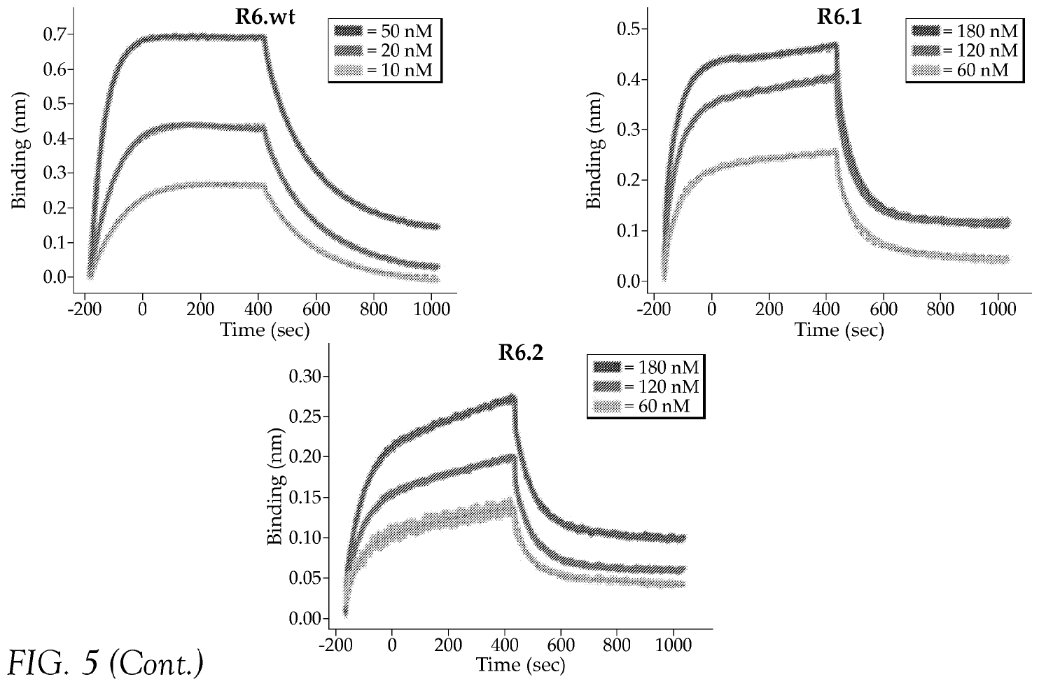

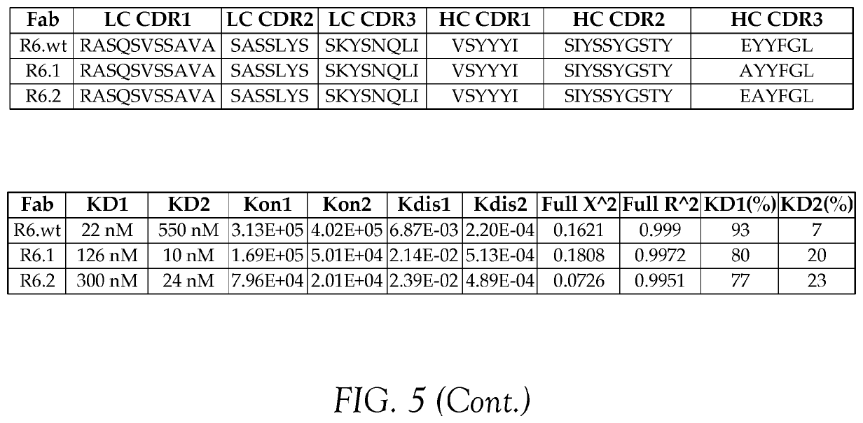

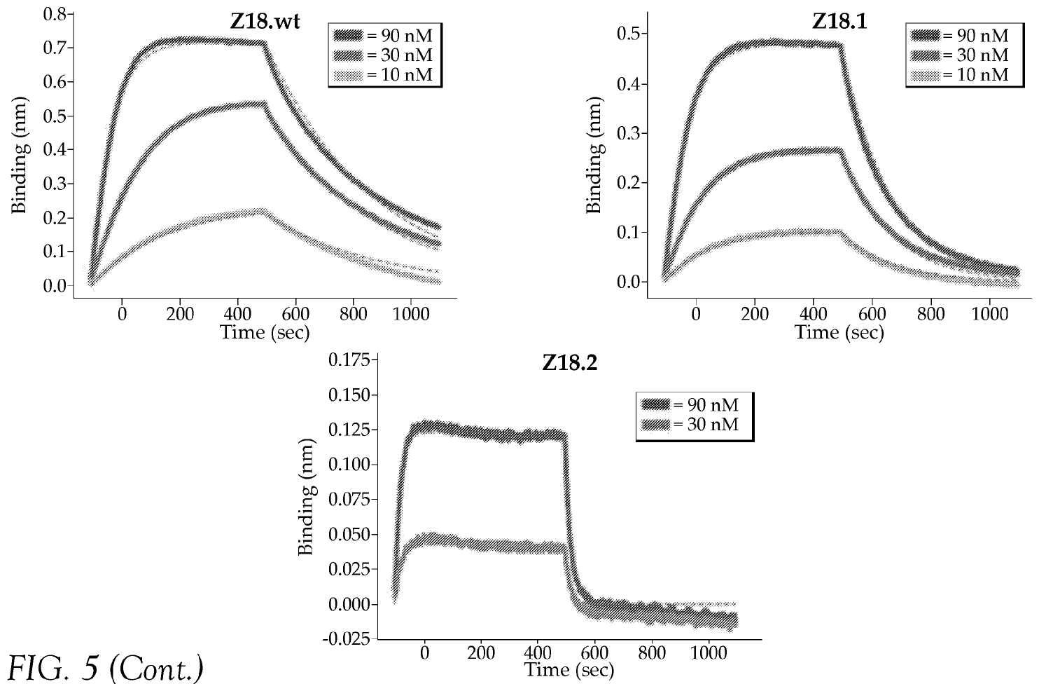

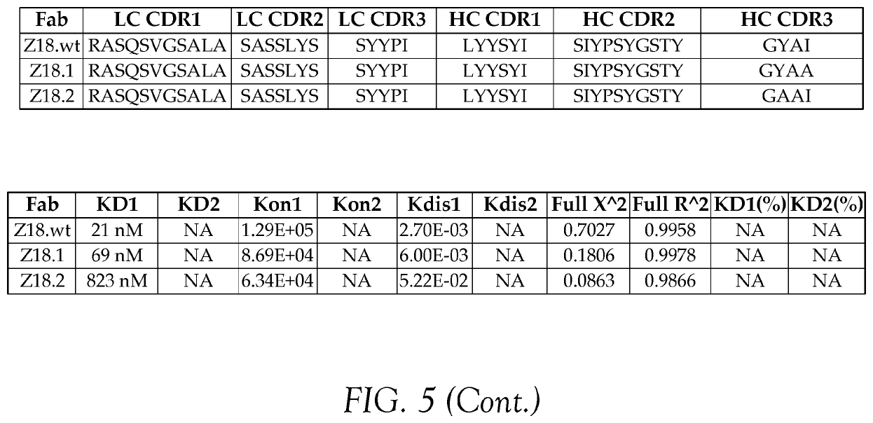

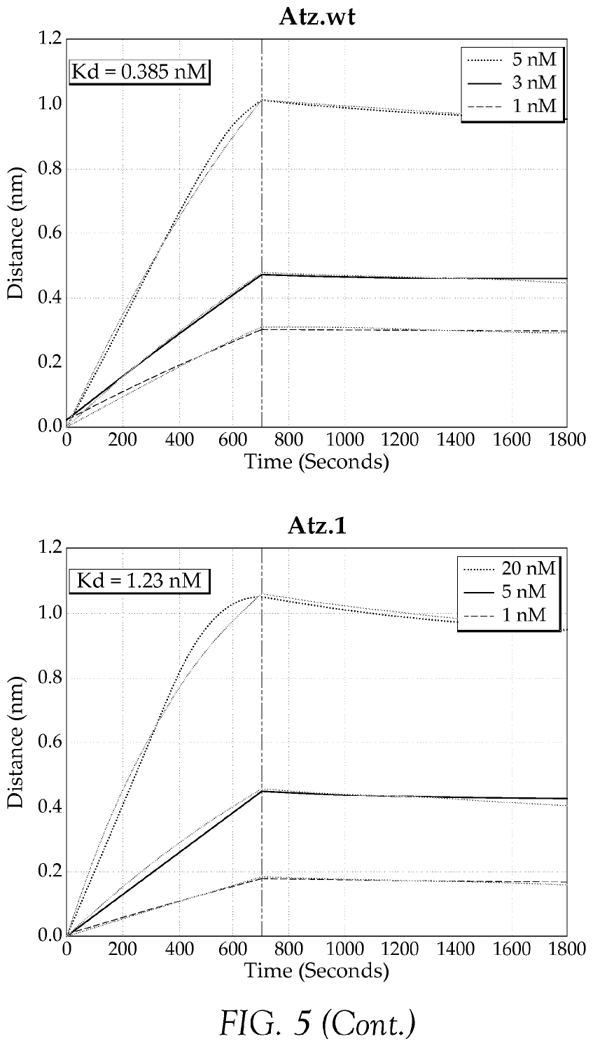

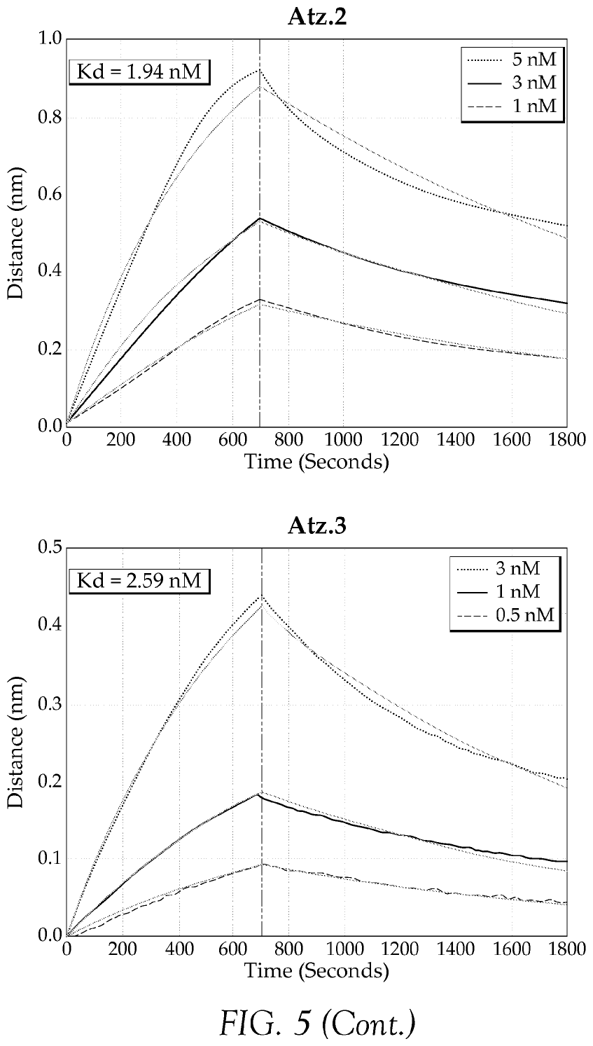

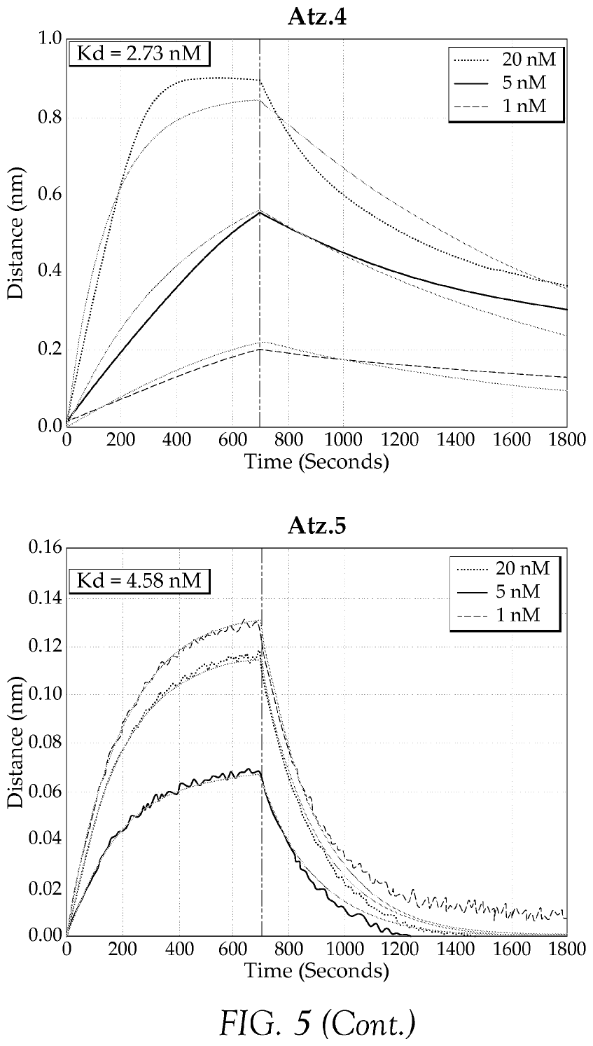

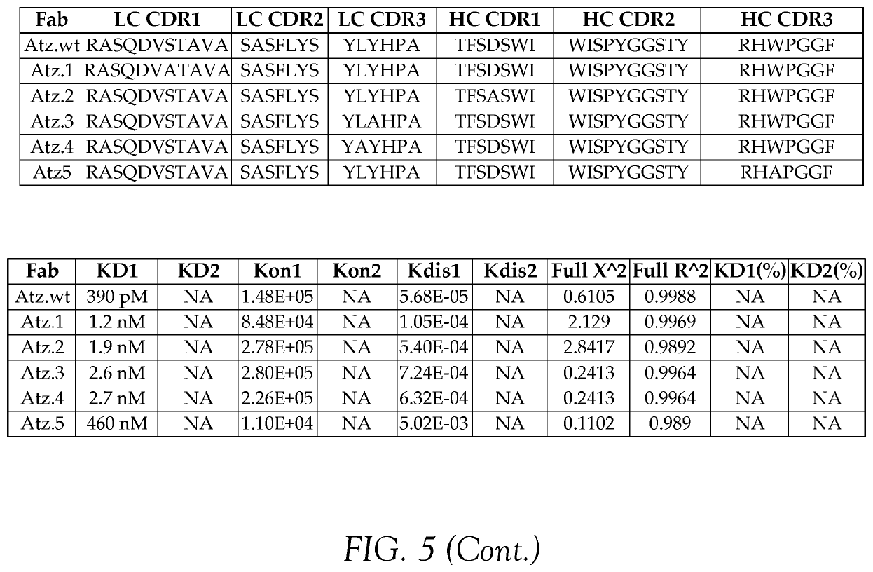

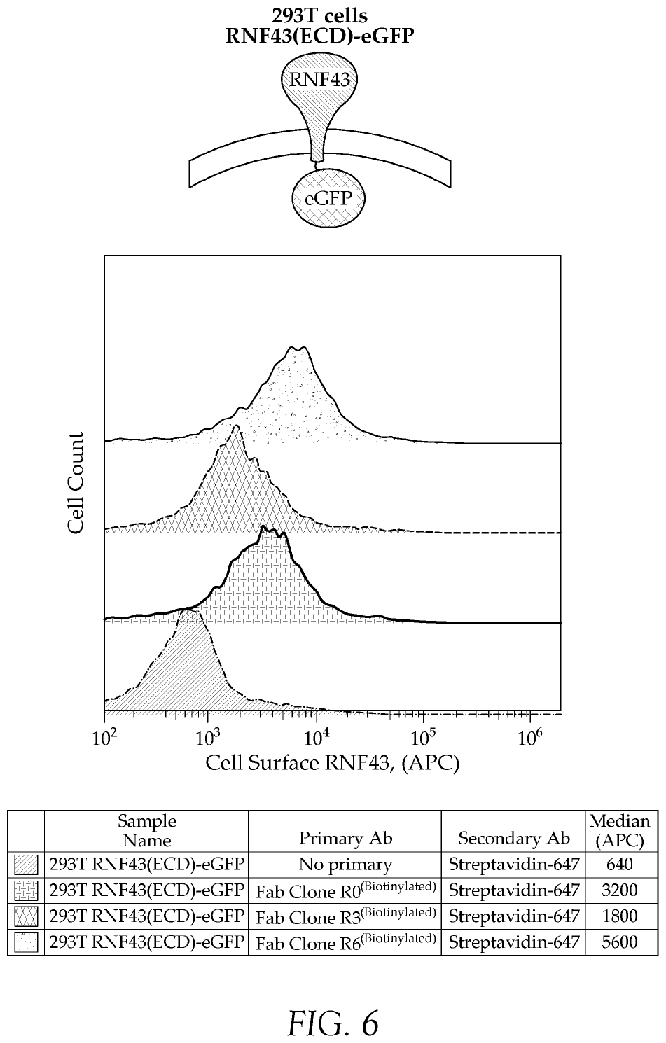

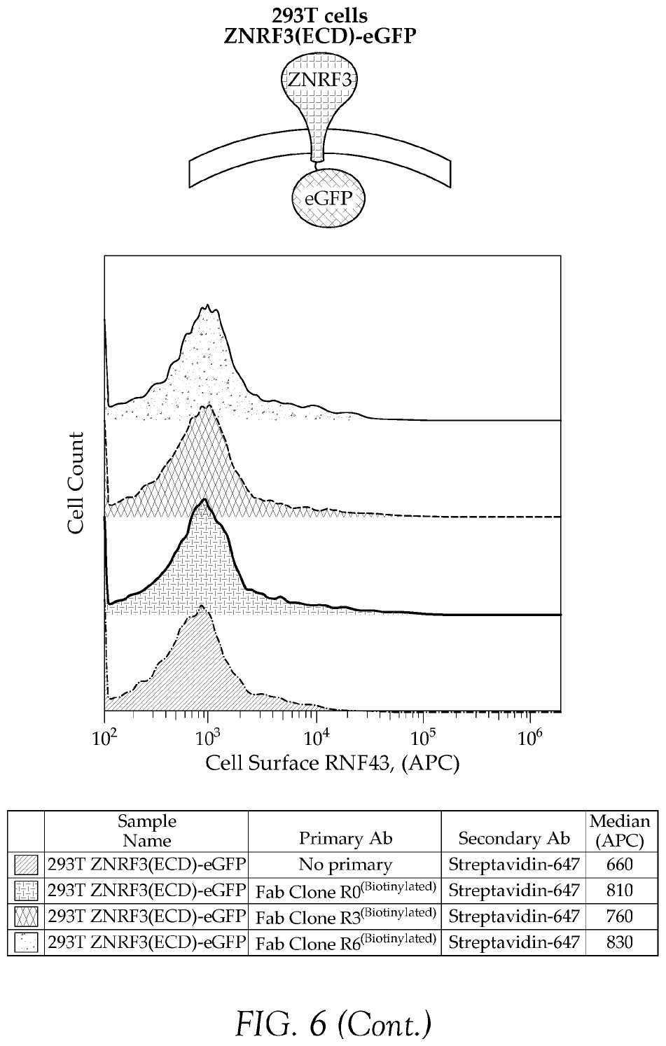

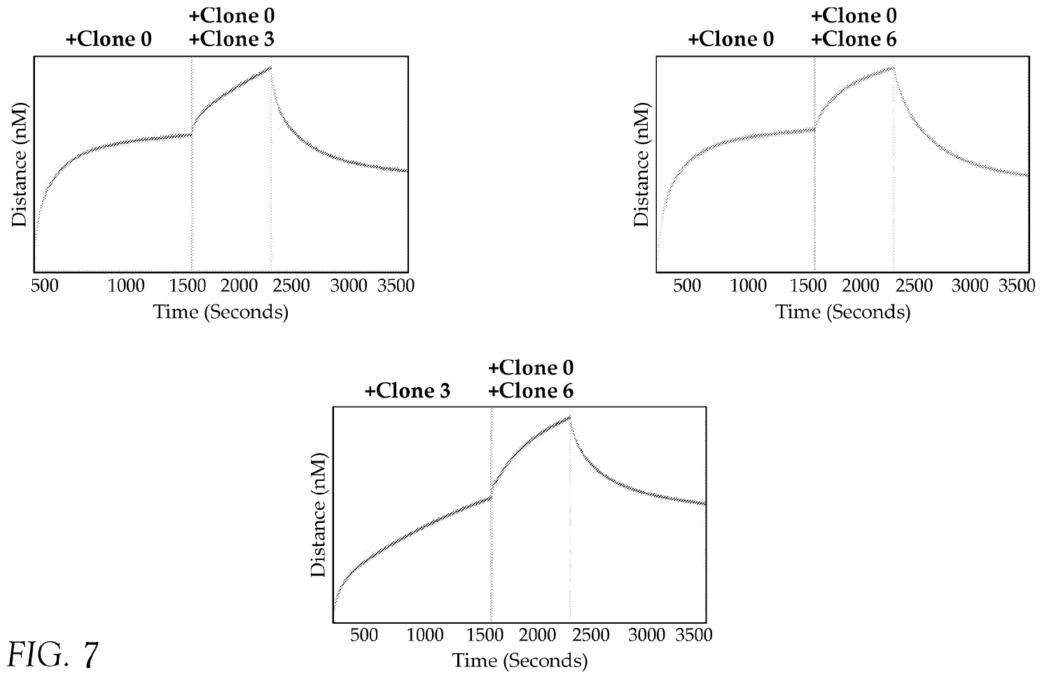

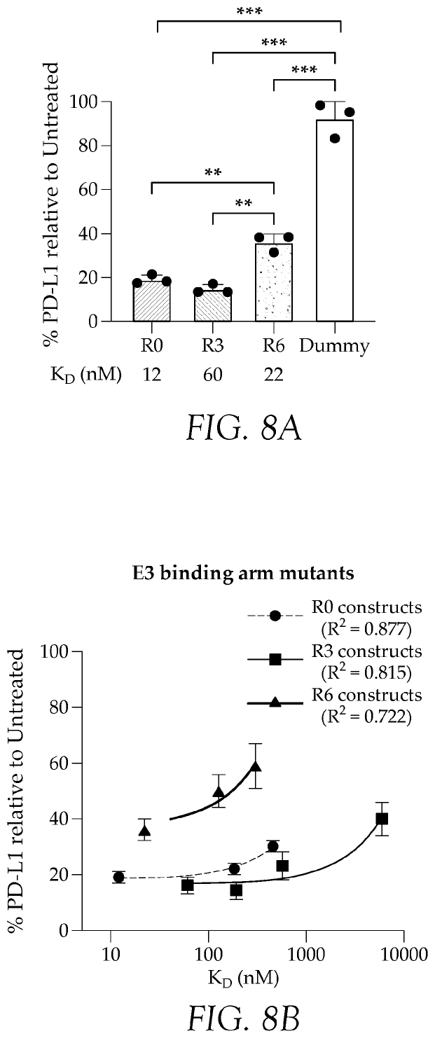

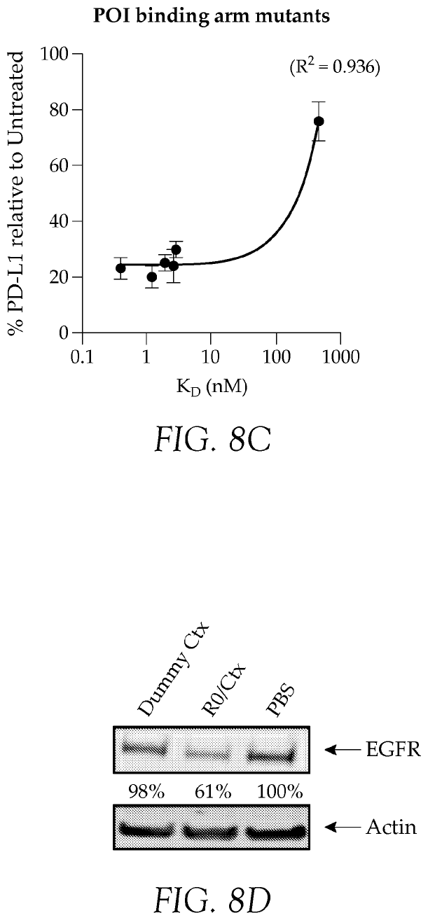

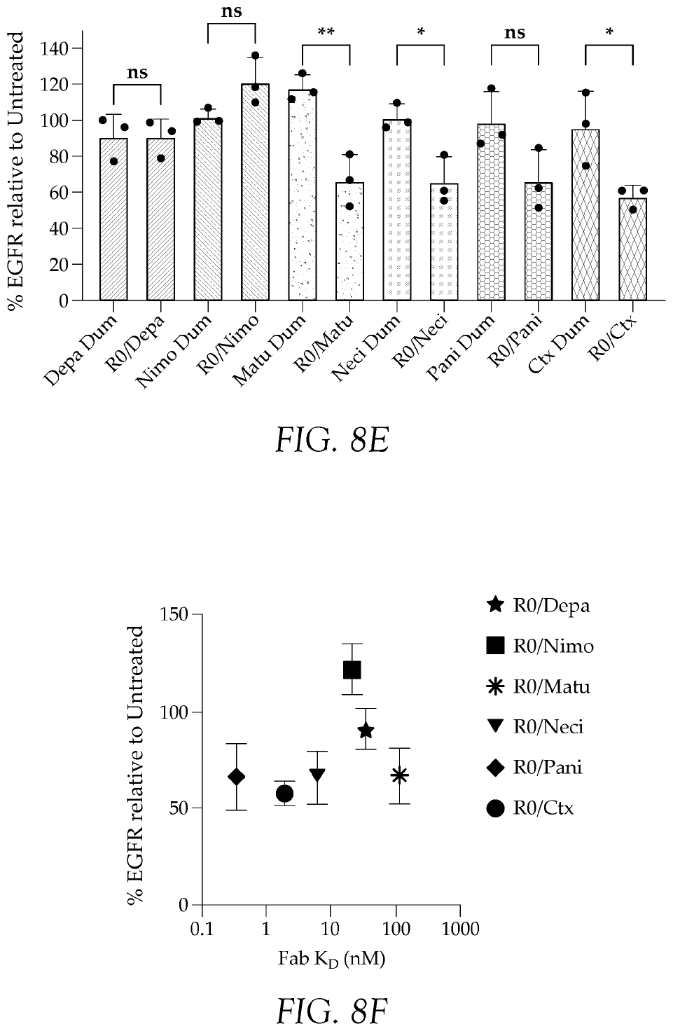

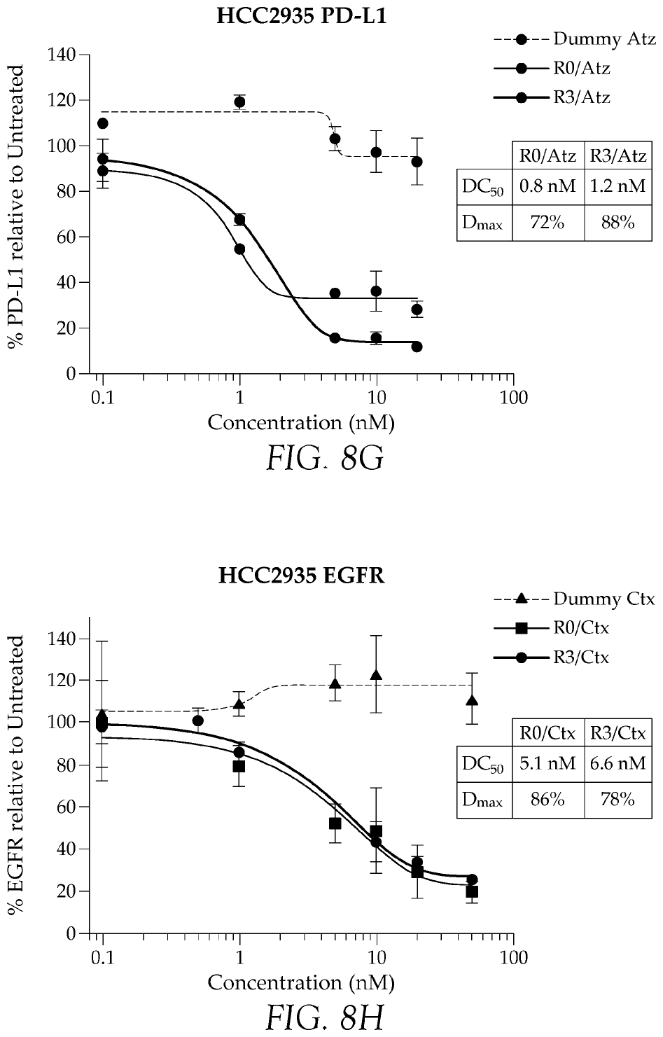

We next sought to interrogate how different binding epitopes of the AbTAC, on either the E3 ligase or POI arm, might affect degradation efficiency. We utilized our in-house Fab phage display library¹¹ to identify additional Fabs that bind non-overlapping RNF43 epitopes compared to our original RNF43 binder, clone R0 (KD: 12.5 nM). We isolated two additional RNF43 binders, clones R3 and R6, that bound to purified RNF43 ectodomain with KD values of 60 nM and 22 nM, respectively, as measured by BLI (FIG. 5). These new clones bound to RNF43 on the surface of cells as assessed by flow-cytometry (FIG. 6). Importantly, each of the three RNF43 Fabs (R0, R3, and R6) bound distinct epitopes as determined by epitope binning by BLI (FIG. 7). Next, we generated R3/Atz and R6/Atz AbTACs as well as an Atz Dummy lacking an E3 ligase binding arm to use as a control for our degradation assays (FIG. 2). We observed PD-L1 degradation for all three AbTACs (R0/Atz, R3/Atz, R6/Atz), but interestingly, there did not appear to be a direct correlation between the affinity of a given E3 epitope binder and the level of degradation. Indeed, the 60 nM affinity binder showed the highest level of degradation, the 22 nM affinity binder showed the lowest level of degradation and the 12 nM binder showed an intermediate level of degradation (FIG. 8A). Taken together, these data indicate that the binding epitope on RNF43 may play a significant role in determining efficient POI degradation.

To directly test how affinity on the degrader arm affects degradation, we sought to systematically reduce the affinity for each of the three RNF43 binders. The heavy chain complementary determining region 3 (CDR3) is typically a major binding element in antibodies. Thus, we performed an alanine scan of the CDR3 of each clone. We were able to generate alanine mutants that incrementally lowered affinities (mostly due to reduced off-rates) from their parental Fabs (FIG. 5). We generated AbTACs for each of these mutants (FIG. 2) and tested these AbTACs for their ability to degrade PD-L1 (FIG. 8B). Decreasing affinity on the RNF43 side correlated with decreased degradation. However, the affinity and corresponding off-rate for RNF43 could be decreased 10-50-fold without major loss of degradation efficiency; this suggests there may be a threshold off-rate, of only modest duration, that is needed to promote efficient degradation.

### Example 4

### Epitope and Affinity Matter for Binding the Target Protein

We next tested how changing the affinity and epitope on the POI arm of the AbTAC might affect degradation. Based on the known structure of atezolizumab in complex with PD-L1, we generated alanine mutations in key interacting residues of atezolizumab's CDRs¹². We chose mutants with a range of affinities (K_{D} varied from 0.3 nM-458 nM) (FIG. 5) and generated the corresponding R0/Atz AbTACs (FIG. 2). These constructs were assayed for their ability to degrade PD-L1, revealing that degradation correlates with the K_{D} of the POI binding arm (R²=0.93) (FIG. 8C). Interestingly, we observed no decrease in degradation between the parental binder (0.3 nM) and the mutants that had a 10-fold lower affinity. This indicates that there may exist a threshold affinity beyond which further degradation is not achieved by simply increasing the affinity of the POI binding arm. However, further decreasing affinity of the POI binding arm to 450 nM almost completely abolished degradation, decreasing it by over 60% compared to the low nM affinity binders. In contrast, decreasing the affinity of the R0, R3 and R6 AbTACs to 450 nM, 5.9 μM, and 300 nM respectively, only abrogated degradation by about 20%. These data suggest that the POI binding arm may be more sensitive to substantial decreases in affinity compared to the E3 binding arms.

We next sought to test how changing the epitope on the POI might affect degradation. However, due to the lack of unique PD-L1 epitope binders, we chose to interrogate a new target EGFR, for which an abundance of different EGFR antibodies and their corresponding epitope binding data and CDR sequences are readily available. We performed an initial degradation assay by generating an AbTAC utilizing the clinically approved cetuximab for the EGFR binding arm, R0/Ctx, as well as a Dummy control (FIG. 2). This construct induced a roughly 50% degradation of EGFR on T24 R-WT cells after 24 hrs as determined by Western blot analysis, confirming that EGFR is a valid target for AbTACs. (FIG. 8D). We next generated five additional R0/EGFR AbTACs and the corresponding Dummy controls (FIG. 2) using EGFR binders known in the literature to bind distinct eiptopes¹³ (Table 1).

TABLE 1
K_{D}s and binding epitopes of EGFR Fabs utilized in this study.

| | Epitope | Fab KD | |
|---|---|---|---|
| Depatuxizumab | Domain II | 34 | nM |
| Nimotuzumab | Domain III (353-358) | 21 | nM |
| Matuzumab | Domain III (460-461) | 113 | nM |
| Necitumumab | Domain III (384-409) | 6.1 | nM |
| Panitumumab | Domain III (386-391) | 340 | pM |
| Cetuximab | Domain III (408-468) | 1.9 | nM |

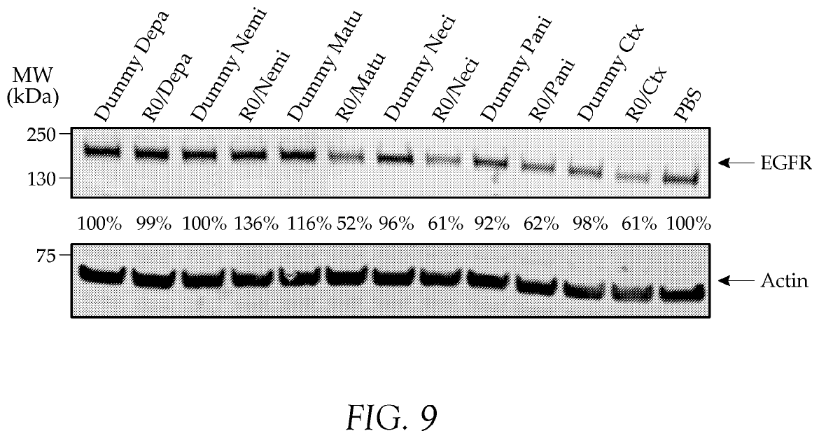

Interestingly, when we tested these constructs for their ability to degrade EGFR on T24 R-WT cells, two AbTACs showed no EGFR degradation while the others degraded EGFR between 40% and 50% as seen for R0/Ctx (FIG. 8E, FIG. 9). As expected, Dummy versions of each EGFR binder did not appear to degrade EGFR (FIG. 8E, FIG. 9). Interestingly, there was little correlation between affinity of the EGFR binding arm and the level of EGFR degradation (FIG. 8F), indicating that the epitope on the POI to which the AbTAC binds also plays a substantial role in degradation, as seen on the degrader arm. Moving these studies to a more relevant cancer cell line, we chose to study HCC2935 cells, an adenocarcinoma tumor cell line, which expresses both PD-L1 and EGFR and exhibits even higher levels of RNF43 expression based on RNA transcript data than T24 WT cells⁹. Indeed, we found that both R0 and R3 based AbTACs could robustly degrade PD-L1 (FIG. 8G) and EGFR (FIG. 8H) to even a greater degree than seen for the engineered T24 R-WT cells.

### Example 5

### Orientation and Valency Affect Degradation Efficiency

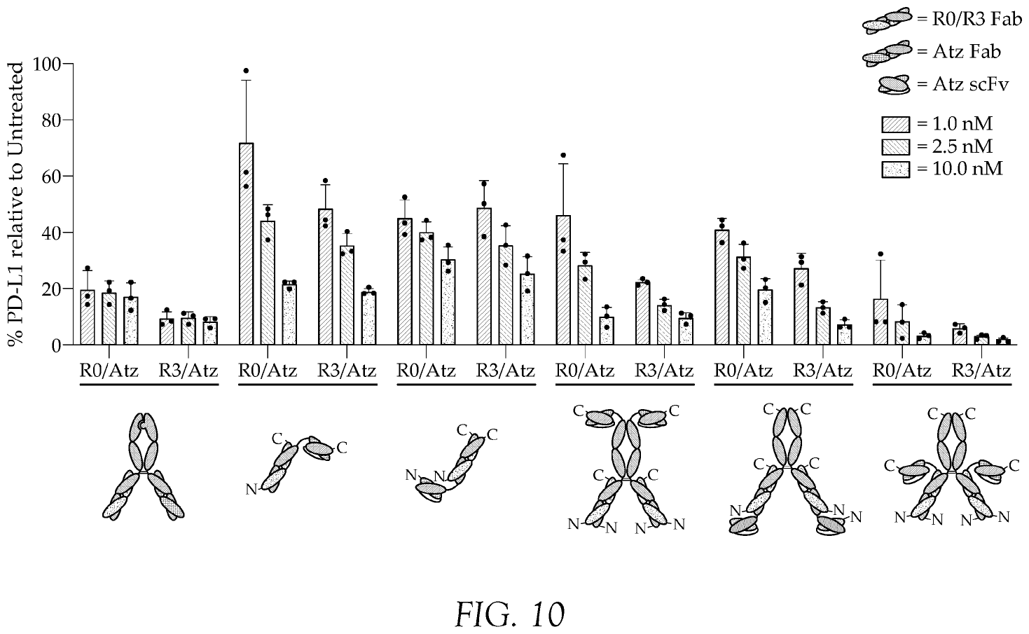

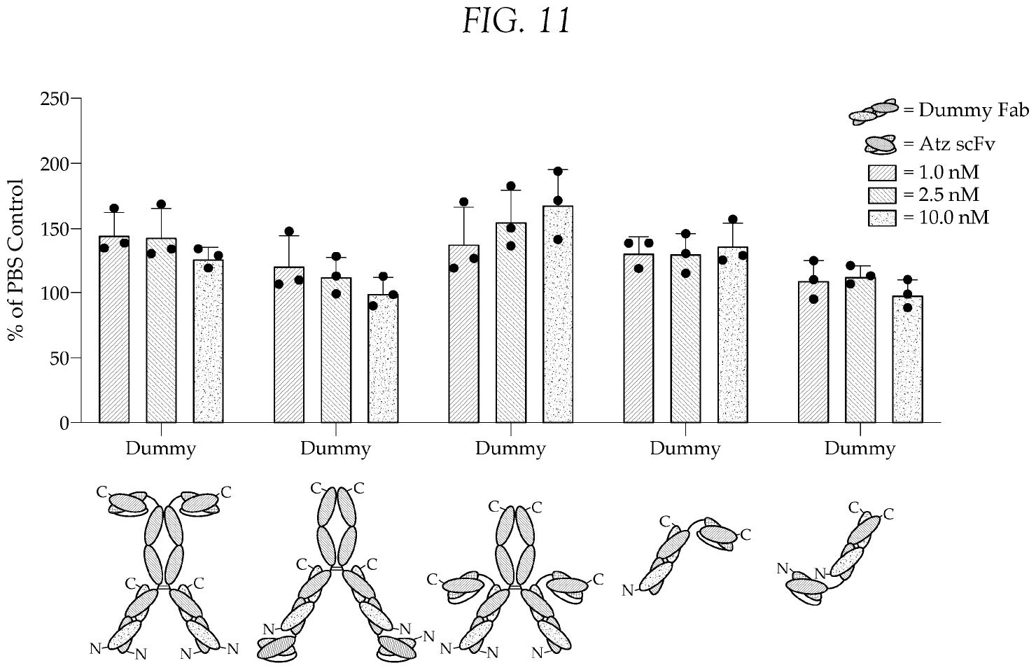

After studying the importance of epitope and affinity in each arm of the AbTAC, we explored how altering the valency, flexibility, and orientation of the AbTAC binding arms affects degradation efficiency (Dmax) and potency (DC50). To allow generation of dual binding antibodies without the worry of light-chain heavy-chain mismatching, we converted Atezolizumab into an scFv binding domain, by fusing the variable domains of the heavy and light chains via a flexible linker. We next generated five different AbTAC constructs for each of the R0 and R3 Fabs, as well as a control Dummy Fab, by fusing an atezolizumab scFv domain to either the N- or C-terminus of a Fab or IgG scaffold (FIG. 2). We then tested the ability of these constructs to degrade PD-L1 on T24 R-WT cells. Gratifyingly, all the R0 and R3 constructs induced significant degradation of PD-L1 (FIG. 10), whereas the dummy constructs did not degrade PD-L1 at all (FIG. 11). For each of the different scaffolds, the R3 based constructs degraded PD-L1 more effectively than the R0 based constructs, similar to what was observed for the original bispecific knob-in-hole AbTACs. Interestingly, the monovalent binding constructs, where the atezolizumab scFv is fused to the N- or C-terminus of the R0 or R3 Fab heavy chain, exhibited worse degradation at each of the tested concentrations than the original bispecific AbTAC constructs. In contrast, the bivalent constructs where the atezolizumab scFv domain is fused to the N-terminus or C-terminus of the R0 or R3 bivalent IgG heavy chain exhibited equitable degradation at the highest concentration tested (10 nM), but noticeably worse degradation at the lowest concentration tested (1 nM) compared to the original bispecific AbTAC constructs. Furthermore, for both the Fab and IgG fusions, the C-terminal scFv fusion was more potent than the corresponding N-terminal fusion. Indeed, the constructs where the scFv is fused to the C-terminus of the light chain degraded PD-L1 the most effectively, exhibiting both the highest Dmax (97% and 99% for R0 and R3 constructs respectively) as well as the most favorable DC50. Taken together, these data indicate that antibody formatting and valency can affect target degradation efficiency, and that AbTACs are both modular and amenable to simplified bivalent dual binding IgG constructs for greater potency.

### Example 6

### Generalization to Another Transmembrane E3 Ligase, ZNRF3

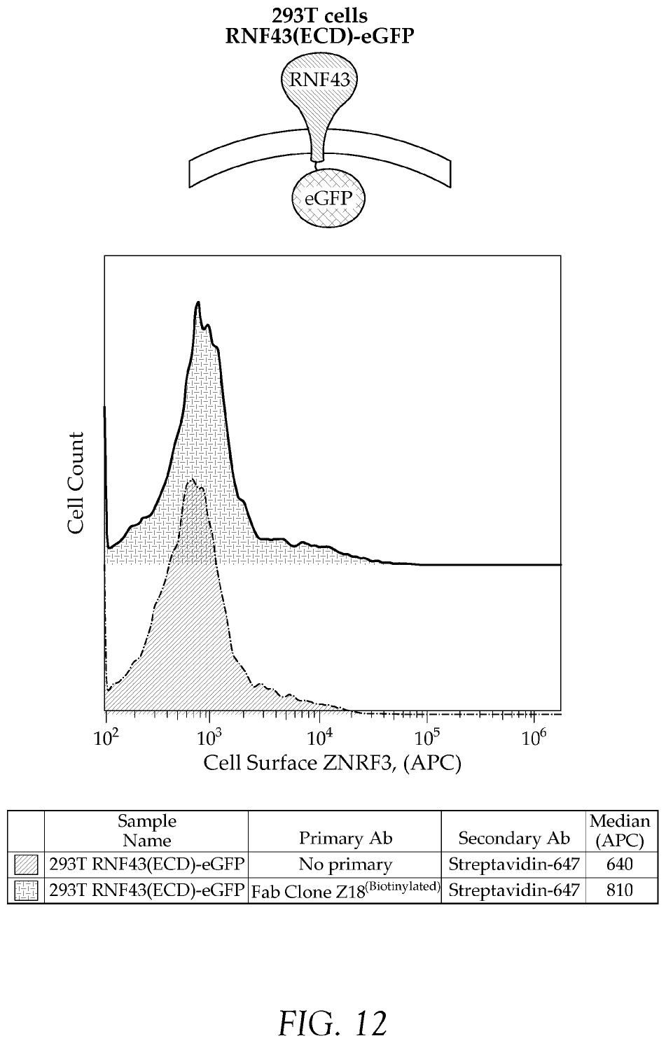

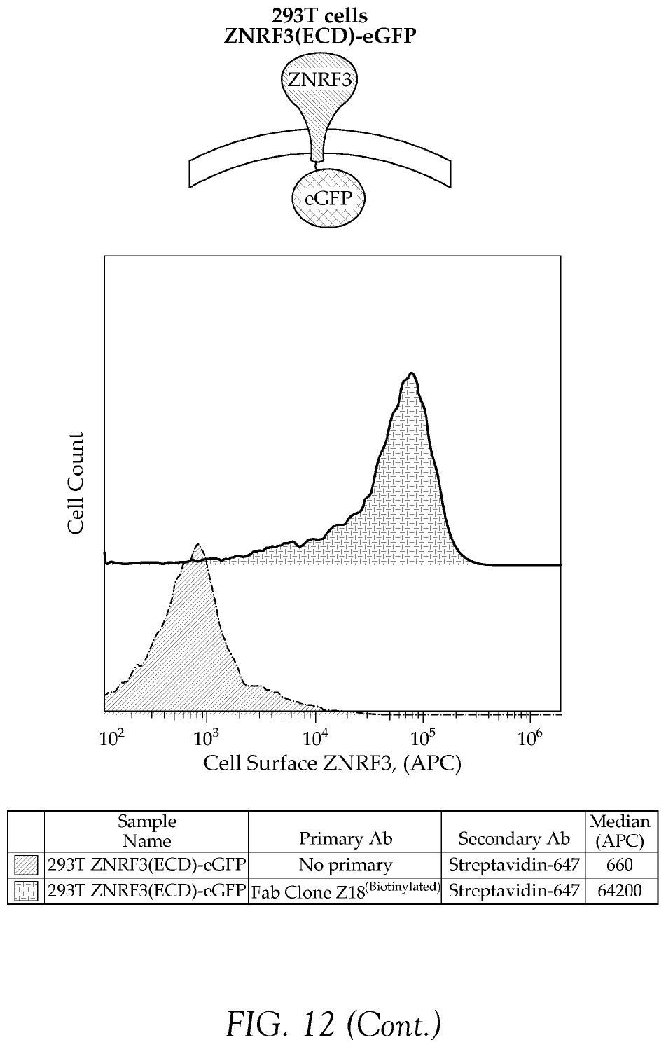

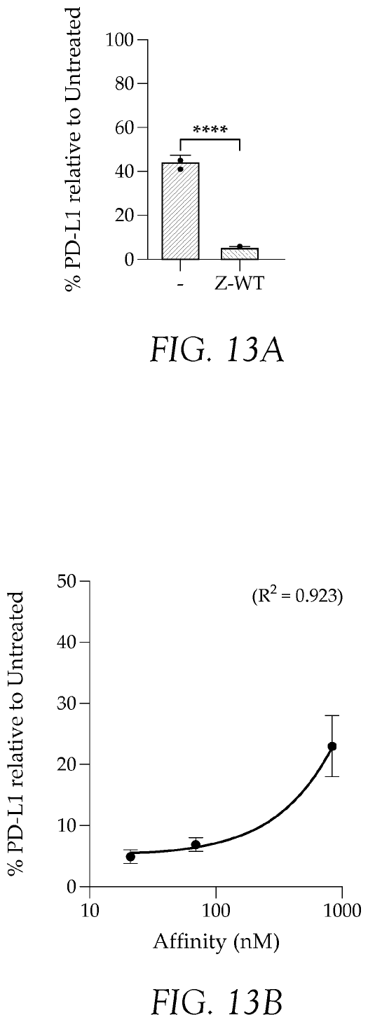

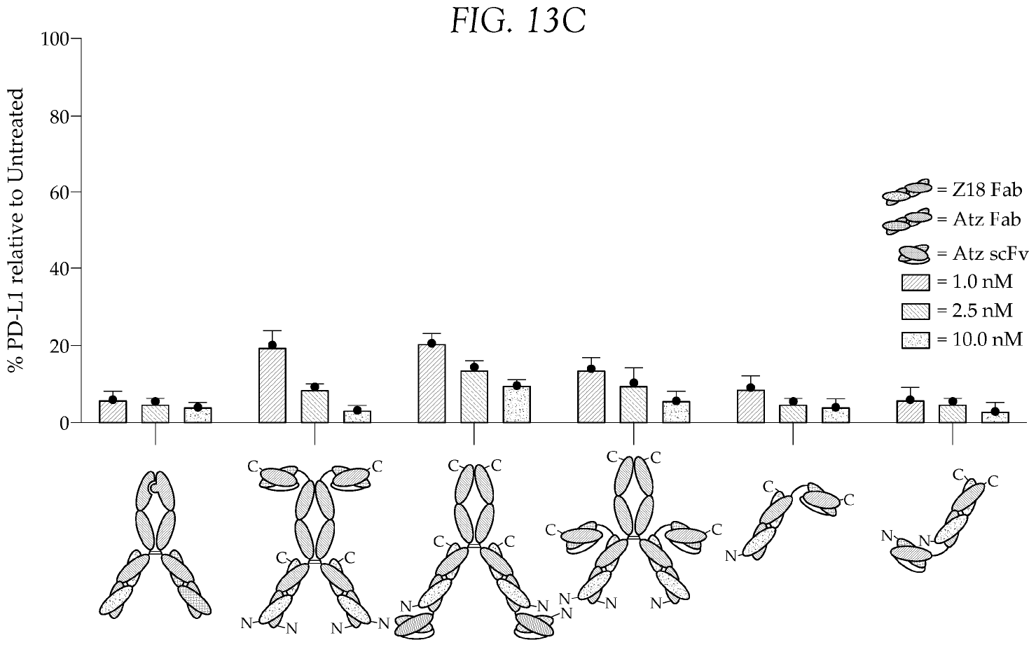

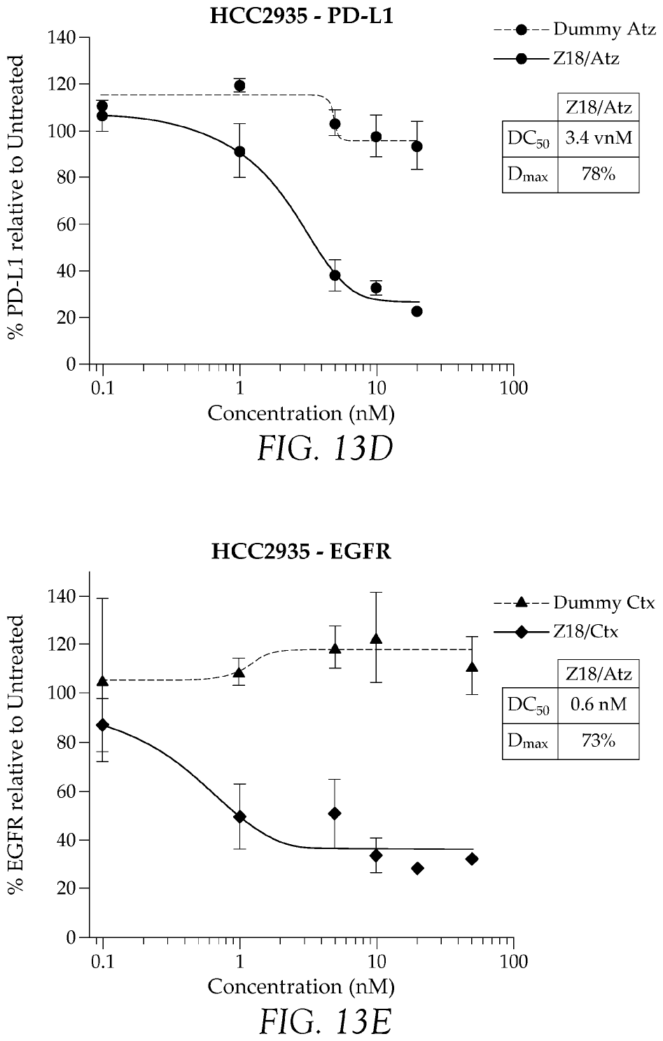

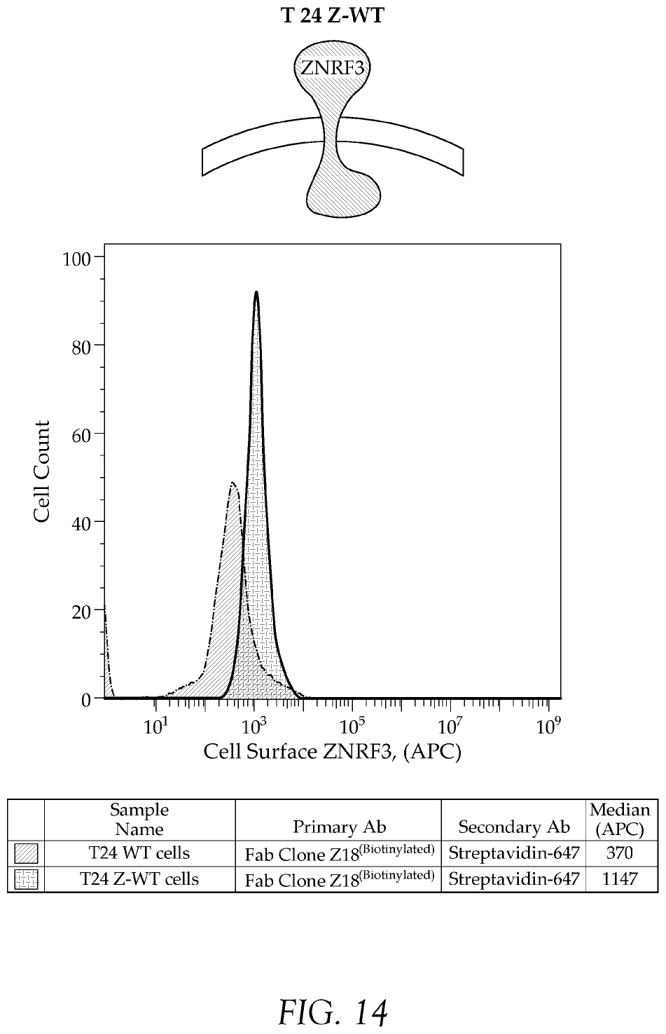

RNF43 is perhaps the best characterized member of the family of transmembrane E3 ligases. Here we sought to test if other members of this family could be used for membrane protein degradation. As such, we turned to the next best characterized and close homolog of RNF43, ZNRF3, another member of the PA-TM-RING family of E3 ligases. Like RNF43, ZNRF3 negatively regulates Wnt signaling by inducing degradation of the membrane receptor Frizzled¹⁴. Importantly, RNF43 and ZNRF3 are often expressed at different levels in different cell lines⁹. As such, ZNRF3 might be more suitable for AbTAC mediated degradation, depending on the cell line being targeted. Utilizing our in-house Fab phage display library, we were able to isolate a Fab, called Z18, that bound ZNRF3 with a KD of 21 nM (FIG. 5). Moreover, Z18 bound specifically to ZNRF3 on the surface of cells expressing ZNRF3, but not to control cells lacking it (FIG. 12). Next, we generated a Z18/Atz AbTAC (FIG. 2) and found that it could degrade roughly 55% of PD-L1 on WT T24 cells (FIG. 13A). To test the degradation dependence for ZNFR3 levels on cells, we generated a stable T24 cell line overexpressing WT ZNRF3 (FIG. 14). Remarkably, the degradation efficiency of PD-L1 increased to 95%, even higher than seen for RNF43 based AbTACs (FIG. 13A).

Next, we applied the same structure-activity relationship (SAR) studies on the ZNRF3 AbTAC as we had for the RNF43 AbTACs. Alanine scanning was used to generate two additional Z18 mutants with affinities for ZNRF3 reduced by about 5- and 50-fold, respectively (FIGS. 2, 5). Similar to what was observed for the RNF43 AbTACs, the affinity of the Z18 AbTACs could be substantially decreased without much loss of degradation efficiency, again indicating that high affinity binding to the degrader arm, ZNRF3, is not required for efficient degradation (FIG. 13B). Next, we generated the same five alternate AbTAC scaffolds used before (FIG. 2) and tested these new constructs for their ability to degrade PD-L1. All constructs were able to robustly degrade PD-L1 (FIG. 13C), showing that Z18 AbTACs are also highly modular and amenable to a variety of different scaffolds. Notably, the mono-valent scFv-Fab fusions were not as potent as the bispecific AbTAC format, while the dual binding IgGs were more potent. This is the same trend that we observed for the alternate RNF43 AbTAC scaffolds. We were also able to apply the Z18/Atz and a Z18/Ctx AbTAC to effectively degrade PD-L1 (FIG. 13D) and EGFR (FIG. 13E) respectively on HCC2935 cancer cells.

### Example 7

### Cellular Mechanisms for RNF43 and Znrf3 AbTACs

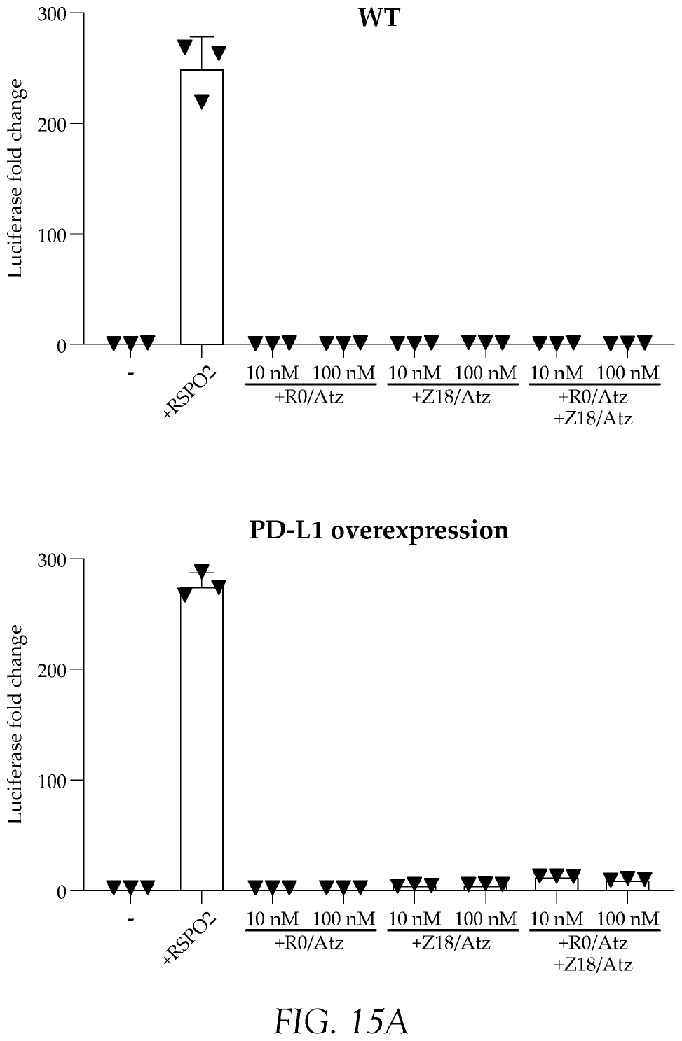

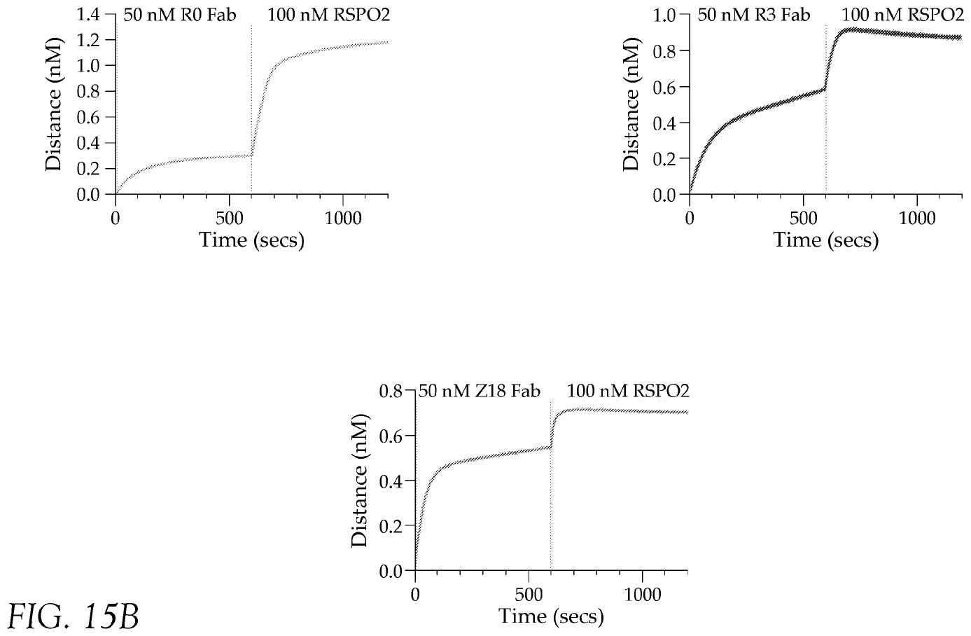

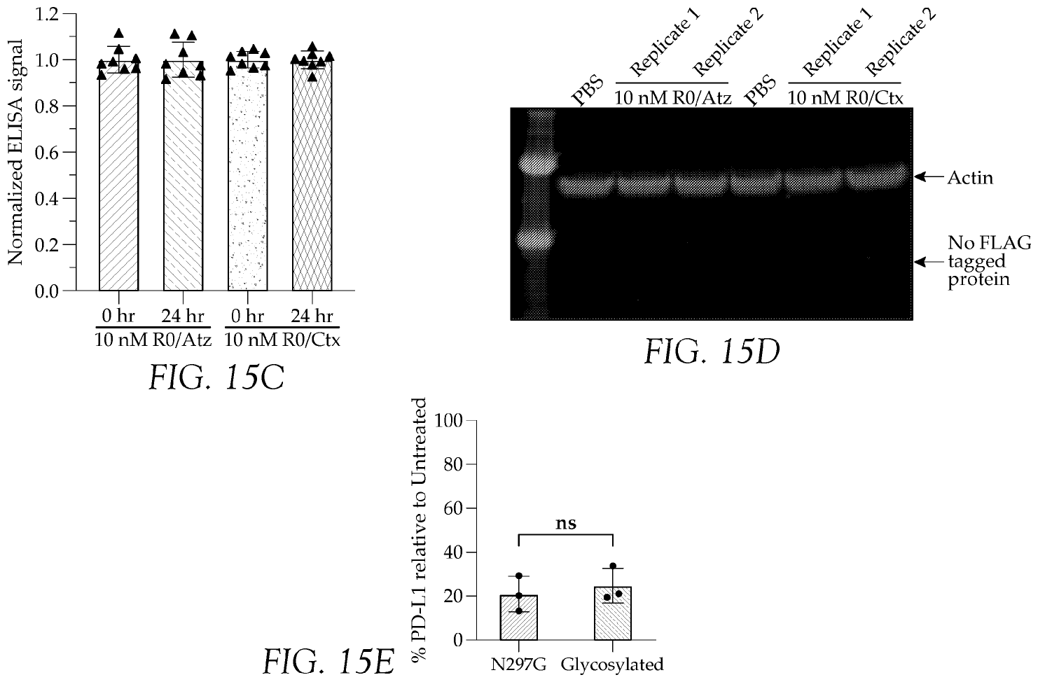

RNF43 and ZNRF3 are naturally recruited by the protein Disheveled to constitutively degrade Frizzled, part of the Wnt receptor complex 15. As such, it was possible that AbTACs could affect the Wnt signaling pathway. To test this, we utilized HEK 293 Super Top Flash (STF) WNT reporter cells (HEK 293 STF), an established luciferase Wnt reporter cell-line¹⁶ Cells were treated with R0/Atz and/or Z18/Atz AbTACs and luciferase activity was measured and compared to treatment with R-spondin (a positive control). Because PD-L1 is not endogenously expressed in these cells, we decided to also perform this assay in cells that were first transfected to over-express PD-L1 to better mimic a typical degradation assay. This would ensure that the AbTAC could bind both the E3 ligase as well as the POI. R-spondin is known to induce the membrane clearance and degradation of the negative regulators of Wnt signaling, RNF43/ZNRF314 (FIG. 15A). Whereas R-spondin treated cells dramatically induced luciferase activity, treatment with 10 nM or 100 nM (10-fold higher than the maximal Dmax) of either AbTAC on its own or in combination had minimal impact on luciferase induction. These data indicate that AbTACs do not potentiate unwanted WNT signaling. Moreover, BLI experiments show that R-spondin maintains the ability to bind RNF43 and ZNRF3 that has been pre-treated with AbTAC, indicating that they bind distinct epitopes (FIG. 15B).

We further studied whether levels of AbTAC in culture with HCC2935 cells were depleted during a typical degradation experiment (10 nM AbTAC for 24 hr at 37° C.). There was no detectable change in the R0/Atz or R0/Ctx AbTAC levels in cell culture as measured by ELISA, suggesting these are not significantly consumed during the experiment (FIG. 15C). Moreover, we did not detect significant gross accumulation and degradation of either AbTAC in HCC2935 cells as measured by Western blot of C-terminally FLAG-tagged versions of the AbTACs (FIG. 15D). N-glycosylation at position 297 in the Fc region is important for binding the FcRγ receptor and for antibody directed cell cytotoxicity¹⁷ (ADCC). We thus expressed in mammalian cells the R0/Atz AbTAC with this glycosylation site or with an N297G mutant. Gratifyingly, we found no change in degradation levels of PD-L1 (FIG. 15E), suggesting that FcRγ receptors are not involved in the targeted degradation we observe.

### Example 8

### Discussion

Targeted protein degradation is coming of age as an important modality for drug discovery. However, although most traditional drugs target extracellular membrane proteins, the focus in the degradation field has largely been on cytosolic targets. Recent developments to co-opt recycling receptors using glycan conjugated antibodies or small molecules have begun to address degradation of extracellular protein targets. Many approaches will be important in this new field of extracellular protein degradation, and we believe AbTACs to have distinct utility. AbTACs are fully genetically encoded and highly modular, which makes them very simple to assemble and test. Furthermore, the bispecific and IgG formats used in this study are well-precedented, which facilitates their therapeutic development.

The focus of our work here has been to understand the physical and cellular parameters that modulate the activity of AbTACs. We observe substantial degradation even when the transcript levels of the target are in large excess to the degrader, suggesting AbTACs might function catalytically. We also show that degradation of the POI can be enhanced for cells containing higher levels of the E3 ligase. This is an important consideration and potential advantage for targeting POIs on specific cell types and could avoid issues of on-target toxicity. We do not observe significant depletion of the AbTAC in cell culture or accumulation or degraded parts in cells by Western blotting. However, we cannot exclude that they may be degraded to some degree. Microscopy studies have previously shown AbTACs traffic to the lysosome, with degradation dependent on lysosomal, and not proteasomal, function⁷. Future studies will be required to map out the detailed trafficking pathways and the fates of intracellular pools while the target is degraded. We further show that AbTACs can be generalized to another transmembrane E3 ligase. This expands the utility to cells that may have low levels of RNF43 but high levels of ZNRF3.

In expanding the use of AbTACs as tools for cell biologists and potential therapeutics, it is critical to understand the SAR of these molecules, which has not been as systematically studied for other targeted protein degradation modalities. AbTACs are readily suited for systematic SAR studies due to their fully recombinant nature allowing rapid genetic optimization of affinity, epitope, orientation and valency. It was surprising to find that only modest binding affinity (K_{D} in mid-nM range) is required for efficient degradation on the degrader arm for either RNF43 and ZNRF3. The fact that most of the change in affinity is from changes in k_{off}, suggests that there is a threshold residence time that is sufficient to support maximal degradation.

Degradation efficiency was highly sensitive to the specific epitopes engaged on both the degrader and the target. We found that three different binders to different epitopes on RNF43 varied in their ability to degrade PD-L1. The importance of epitope was further demonstrated on the POI side through different binders to the EGFR target. Six commercial EGFR antibodies with varying affinities and epitopes showed differential degradation efficiencies. Interestingly, we observed that while the R3 based AbTACs appeared to degrade PD-L1 more robustly than the R0 based AbTACs, both R3 and R0 based AbTACs appeared to degrade EGFR similarly when Ctx was used as the EGFR binding arm. This may indicate the importance of epitope in the larger context of complex formation that occurs between the POI and E3 ligase. As such, the specific combination of E3 ligase and POI binders employed will likely be an important consideration in terms of achieving maximal degradation. Overall, these studies suggest that AbTAC binding epitopes for both the E3 ligase and POI are important determinants of degradation efficiency.

We were able to explore the role of orientation and valency using the bispecific knob-in-hole IgG format as well as monovalent or bi-valent IgG formats to generate tandem dual binders. These bispecific IgG AbTACs were better degraders than the monovalent tandem binders, despite them both utilizing the same arms to engage the degrader and POI. There is a large difference in spacing and orientation between the bispecific IgG and tandem binder, which we believe rationalizes this finding. However, one of the dual binding IgG formats worked even better than the original bispecific knob-in-hole IgG, suggesting that valency can improve the degradation efficiency. Furthermore, the placement of the binding arms within the IgG scaffold also affected degradation efficiency, confirming that spacing of the binding arms matters. While we do not have structural data of these complexes, it is well known from vast literature on bispecific T-cell engagers¹⁸ as well as bi-paratopic binders to Her2¹⁹ and the SARS-CoV-2 spike protein²⁰ that valency, orientation and proximity are all critical factors for effective functional engagement.

In summary, these studies help to expand our mechanistic understanding of AbTAC mediated degradation. Our SAR studies revealed the importance of epitope, affinity, orientation, and valency leading to increases in Dₘₐₓ up to 95. In addition to RNF43, we validate a new transmembrane E3 ligase, ZNRF3, which provides flexibility to the platform and potential cell type specificity. It will be interesting to determine in future experiments whether AbTACs can be applied to target more complex cell surface proteins such as multi-pass transmembrane receptors. Furthermore, the functional consequence of AbTAC mediated degradation of these membrane receptors will need to be probed. Taken together, we believe this work provides a roadmap for the development of future AbTACs, thereby expanding their utility for targeted cell surface protein degradation.

### REFERENCES

(1) Bond, M. J.; Crews, C. M. Proteolysis Targeting Chimeras (PROTACs) Come of Age: Entering the Third Decade of Targeted Protein Degradation. RSC Chem. Biol. 2021, 2 (3), 725-742. https://doi.org/10.1039/d1cb00011j.
(2) Pettersson, M.; Crews, C. M. PROteolysis TArgeting Chimeras (PROTACs)-Past, Present and Future. Drug Discov. Today Technol. 2019, 31, 15-27. https://doi.org/10.1016/j.ddtec.2019.01.002.
(3) Békés, M.; Langley, D. R.; Crews, C. M. PROTAC Targeted Protein Degraders: The Past Is Prologue. Nat. Rev. Drug Discov. 2022, 21 (3), 181-200. https://doi.org/10.1038/s41573-021-00371-6.
(4) Banik, S. M.; Pedram, K.; Wisnovsky, S.; Ahn, G.; Riley, N. M.; Bertozzi, C. R. Lysosome-Targeting Chimaeras for Degradation of Extracellular Proteins. Nature 2020, 584 (7820), 291-297. https://doi.org/10.1038/s41586-020-2545-9.
(5) Ahn, G.; Banik, S. M.; Miller, C. L.; Riley, N. M.; Cochran, J. R.; Bertozzi, C. R. LYTACs That Engage the Asialoglycoprotein Receptor for Targeted Protein Degradation. Nat. Chem. Biol. 2021, 17 (9), 937-946. https://doi.org/10.1038/s41589-021-00770-1.
(6) Zhou, Y.; Teng, P.; Montgomery, N. T.; Li, X.; Tang, W. Development of Triantennary N-Acetylgalactosamine Conjugates as Degraders for Extracellular Proteins. ACS Cent. Sci. 2021, 7 (3), 499-506. https://doi.org/10.1021/acscentsci.1c00146.
(7) Cotton, A. D.; Nguyen, D. P.; Gramespacher, J. A., Seiple, I. B.; Wells, J. A. Development of Antibody-Based PROTACs for the Degradation of the Cell-Surface Immune Checkpoint Protein PD-L1. J. Am. Chem. Soc. 2021, 143 (2), 593-598. https://doi.org/10.1021/jacs.0c10008.
(8) Chames, P.; Van Regenmortel, M.; Weiss, E.; Baty, D. Therapeutic Antibodies: Successes, Limitations and Hopes for the Future. Br. J. Pharmacol. 2009, 157 (2), 220-233. https://doi.org/10.1111/j.1476-5381.2009.00190.x.
(9) Papatheodorou, I.; Moreno, P.; Manning, J.; Fuentes, A. M.-P.; George, N.; Fexova, S.; Fonseca, N. A.; Fullgrabe, A.; Green, M.; Huang, N.; Huerta, L.; et al. Expression Atlas Update: From Tissues to Single Cells. Nucleic Acids Res. 2020, 48 (D1), D77-D83. https://doi.org/10.1093/nar/gkz947.
(10) Ridgway, J. B.; Presta, L. G.; Carter, P. “Knobs-into-Holes” Engineering of Antibody CH3 Domains for Heavy Chain Heterodimerization. Protein Eng. 1996, 9 (7), 617-621. https://doi.org/10.1093/protein/9.7.617.
(11) Hornsby, M.; Paduch, M.; Miersch, S.; Sääf, A.; Matsuguchi, T.; Lee, B.; Wypisniak, K.; Doak, A.; King, D.; Usatyuk, S.; et al. High Through-Put Platform for Recombinant Antibodies to Folded Proteins. Mol. Cell. Proteomics MCP 2015, 14 (10), 2833-2847. https://doi.org/10.1074/mcp.0115.052209.
(12) Zhang, F.; Qi, X.; Wang, X.; Wei, D.; Wu, J.; Feng, L.; Cai, H.; Wang, Y.; Zeng, N.; Xu, T.; et al. Structural Basis of the Therapeutic Anti-PD-L1 Antibody Atezolizumab. Oncotarget 2017, 8 (52), 90215-90224. https://doi.org/10.18632/oncotarget.21652.
(13) Cai, W.-Q.; Zeng, L.-S.; Wang, L.-F.; Wang, Y.-Y.; Cheng, J.-T.; Zhang, Y.; Han, Z.-W.; Zhou, Y.; Huang, S.-L.; Wang, X.-W.; et al. The Latest Battles Between EGFR Monoclonal Antibodies and Resistant Tumor Cells. Front. Oncol. 2020, 10, 1249. https://doi.org/10.3389/fonc.2020.01249.
(14) Hao, H.-X.; Xie, Y.; Zhang, Y.; Charlat, O.; Oster, E.; Avello, M.; Lei, H.; Mickanin, C.; Liu, D.; Ruffner, H.; et al. ZNRF3 Promotes Wnt Receptor Turnover in an R-Spondin-Sensitive Manner. Nature 2012, 485 (7397), 195-200. https://doi.org/10.1038/nature11019.
(15) Zeng, C.-M.; Chen, Z.; Fu, L. Frizzled Receptors as Potential Therapeutic Targets in Human Cancers. Int. J. Mol. Sci. 2018, 19 (5), E1543. https://doi.org/10.3390/ijms19051543.
(16) Thorne, C. A.; Hanson, A. J.; Schneider, J.; Tahinci, E.; Orton, D.; Cselenyi, C. S.; Jernigan, K. K.; Meyers, K. C.; Hang, B. I.; Waterson, A. G.; et al. Small-Molecule Inhibition of Wnt Signaling through Activation of Casein Kinase 1α. Nat. Chem. Biol. 2010, 6 (11), 829-836. https://doi.org/10.1038/nchembio.453.
(17) Borrok, M. J.; Jung, S. T.; Kang, T. H.; Monzingo, A. F.; Georgiou, G. Revisiting the Role of Glycosylation in the Structure of Human IgG Fc. ACS Chem. Biol. 2012, 7 (9), 1596-1602. https://doi.org/10.1021/cb300130k.
(18) Huehls, A. M., Coupet, T. A.; Sentman, C. L. Bispecific T-Cell Engagers for Cancer Immunotherapy. Immunol. Cell Biol. 2015, 93 (3), 290-296. https://doi.org/10.1038/icb.2014.93.
(19) Kast, F.; Schwill, M.; Stüber, J. C.; Pfundstein, S.; Nagy-Davidescu, G.; Rodríguez, J. M. M.; Seehusen, F.; Richter, C. P.; Honegger, A.; Hartmann, K. P.; et al. Engineering an Anti-HER2 Biparatopic Antibody with a Multimodal Mechanism of Action. Nat. Commun. 2021, 12 (1), 3790. https://doi.org/10.1038/s41467-021-23948-6.
(20) Bracken, C. J.; Lim, S. A.; Solomon, P.; Rettko, N. J.; Nguyen, D. P.; Zha, B. S.; Schaefer, K.; Byrnes, J. R.; Zhou, J.; Lui, I.; et al. Bi-Paratopic and Multivalent VH Domains Block ACE2 Binding and Neutralize SARS-COV-2i. Nat. Chem. Biol. 2021, 17 (1), 113-121. https://doi.org/10.1038/s41589-020-00679-1.
(21) Garrett, T. P. J.; Burgess, A. W.; Gan, H. K.; Luwor, R. B.; Cartwright, G.; Walker, F.; Orchard, S. G.; Clayton, A. H. A.; Nice, E. C.; Rothacker, J.; et al. Antibodies Specifically Targeting a Locally Misfolded Region of Tumor Associated EGFR. Proc. Natl. Acad. Sci. U.S.A. 2009, 10⁶ (13), 5082-5087. https://doi.org/10.1073/pnas.0811559106.
(22) Talavera, A.; Friemann, R.; Gómez-Puerta, S.; Martinez-Fleites, C.; Garrido, G.; Rabasa, A.; López-Requena, A.; Pupo, A.; Johansen, R. F.; Sánchez, O.; Krengel, U.; et al. Nimotuzumab, an Antitumor Antibody That Targets the Epidermal Growth Factor Receptor, Blocks Ligand Binding While Permitting the Active Receptor Conformation. Cancer Res. 2009, 69 (14), 5851-5859. https://doi.org/10.1158/0008-5472.CAN-08-4518.
(23) Schmiedel, J.; Blaukat, A.; Li, S.; Knöchel, T.; Ferguson, K. M. Matuzumab Binding to EGFR Prevents the Conformational Rearrangement Required for Dimerization. Cancer Cell 2008, 13 (4), 365-373. https://doi.org/10.1016/j.ccr.2008.02.019.
(24) Bagchi, A.; Haidar, J. N.; Eastman, S. W.; Vieth, M.; Topper, M.; Iacolina, M. D.; Walker, J. M.; Forest, A.; Shen, Y.; Novosiadly, R. D.; et al. Molecular Basis for Necitumumab Inhibition of EGFR Variants Associated with Acquired Cetuximab Resistance. Mol. Cancer Ther. 2018, 17 (2), 521-531. https://doi.org/10.1158/1535-7163.MCT-17-0575.
(25) Sickmier, E. A.; Kurzeja, R. J. M.; Michelsen, K.; Vazir, M.; Yang, E.; Tasker, A. S. The Panitumumab EGFR Complex Reveals a Binding Mechanism That Overcomes Cetuximab Induced Resistance. PloS One 2016, 11 (9), e0163366. https://doi.org/10.1371/journal.pone.0163366.

What is claimed:

1. A dual binding agent comprising:
a) a first binding domain that specifically binds to a E3 ligase, wherein the first binding domain comprises an IgG scaffold; and
b) a second binding domain that specifically binds to an extracellular epitope on a target protein of a target cell, wherein the second binding domain is fused to the C-terminus of the light chain of the IgG scaffold, and wherein both the E3 ligase and the target protein are membrane associated.

2. A dual binding agent comprising:
a) a first binding domain that specifically binds to a E3 ligase, wherein the first binding domain comprises an IgG scaffold; and
b) a second binding domain that specifically binds to an extracellular epitope on a target protein of a target cell, wherein the second binding domain is fused to the C-terminus of the heavy chain of the IgG scaffold, and wherein both the E3 ligase and the target protein are membrane associated.

3. A dual binding agent comprising:
a) a first binding domain that specifically binds to a E3 ligase, wherein the first binding domain comprises an IgG scaffold; and
b) a second binding domain that specifically binds to an extracellular epitope on a target protein of a target cell, wherein the second binding domain is fused to the N-terminus of the heavy chain of the IgG scaffold, and wherein both the E3 ligase and the target protein are membrane associated.

4. The dual binding agent of any preceding claim, wherein binding of the dual binding agent to both the E3 ligase and the target protein results in ubiquitination and/or internalization of the target protein.

5. The dual binding agent of any preceding claim, wherein the target cell is a neoplastic cell.

6. The dual binding agent of any preceding claim, wherein the target protein is selected from the group consisting of PD-L1, PD-1, CTLA-4, A2AR, B7-H3, B7-H4, BTLA, KIR, LAG3, NKG2D, TIM-3, VISTA, and SIGLEC7.

7. The dual binding agent of any preceding claim, wherein the first binding domain specifically binds to an extracellular protein attached to an E3 ligase or a transmembrane protein that interacts with an E3 ligase.

8. The dual binding agent of any preceding claim, wherein the dual binding agent induces degradation of the target protein with a Dₘₐₓ of at least 20%.

9. The dual binding agent of any preceding claim, wherein degradation of the target protein reduces the ability of the target cell to proliferate.

10. The dual binding agent of any preceding claim, wherein the target protein is selected from the group consisting of HER2, CD19, CD20, CDCP1, PD-L1, EGFR, MMP14, and CTLA-4.

11. The dual binding agent of any preceding claim, wherein the E3 ligase is a transmembrane protein.

12. The dual binding agent of claim 11, wherein the E3 ligase is selected from the group consisting of RNF43, ZNRF3, RNF133, RNF148, GRAIL (RNF128), RNF149, Goliath (RNF130), RNF150, RNF122, ZNRF4, RNF13, RNF167, RNF121, RNF175, DCST1, March6, Kf-1 (RNF10³), RNF182, RNF145, TRC8 (RNF139), HRD1 (SYVN1), RNFT1, MAPL (MUL1), RNF152, RNF26, RINES (RNF180), MARCHF3, MARCHF2, MARCHF8, MARCH1 (MARCHF1), March11, MARCHF9, March4, RNF186, RNF170, RNF185, RMA1 (RNF5), TRIM59 (RNF10⁴), TRIM13, MARCHF5 (MARCH5), RNF197 (CGRF1), RNF183, RNF217, RNF144B, RNF144A, RNF19B, and RNF19A.

13. The dual binding agent of any preceding claim, wherein the second binding domain is selected from the group consisting of an sc-Fv, single-domain antibodies, nanobodies, Fabs, monospecific Fab2, Fc, minibodies, IgNAR, V-NAR, hcIgG, VHH domains, camelid antibodies, peptibodies, DARPins, and a small molecule.

14. A nucleic acid that encodes the dual binding agent of any one of claims 1 to 13.

15. The nucleic acid of claim 14, wherein the nucleic acid is operably connected to a promoter.

16. An engineered cell comprising the nucleic acid of claim 14.

17. The engineered cells of claim 16, wherein the cell is a B cell, a B memory cell, or a plasma cell.

18. A method for making a dual binding agent, the method comprising:
a) introducing into a host cell one or more of the nucleic acid(s) of claim 14;
b) culturing the host cell of step (a) and
c) inducing expression of the dual binding agent.

19. A vector, comprising the nucleic acid of claim 14.

20. A pharmaceutical composition, comprising: (1) the dual binding agent of any one of claims 1 to 13, the nucleic acid of claim 14, and (2) a pharmaceutically acceptable carrier.

21. A method of treating a neoplastic disease or disorder in a subject, the method comprising administering to a subject in need thereof, a therapeutically effective amount of:
a) the dual binding agent of any one of claims 1 to 13;
b) the nucleic acid of any one of claims 14-15; or
c) the cell of any one of claims 16-17.

22. A use for the treatment of neoplastic disease of:
a) the dual binding agent of any one of claims 1-13;
b) the nucleic acid of any one of claims 14-15; or
c) the cell of any one of claims 16-17.

23. A use for the manufacture of a medicament for the treatment of neoplastic disease of:
a) the dual binding agent of any one of claims 1-13;
b) the nucleic acid of any one of claims 14-15; or
c) the cell of any one of claims 16-17.


SEQUENCE LISTING (17 sequences)

SEQ ID NO: 1 - protein, 458 aa, synthetic construct
EISEVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 453 aa, synthetic construct
EISEVQLVESGGGLVQPGGSLRLSCAASGFNIYYYSIHWVRQAPGKGLEWVASIYSSSGYTSYADSVKGRFTISADTSKNTAYLQMNSLRAEDTAVYYCARYPYWYFDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 448 aa, synthetic construct
EISEVQLVESGGGLVQPGGSLRLSCAASGFNLYYSYIHWVRQAPGKGLEWVASIYPSYGSTYYADSVKGRFTISRDNSKNTLYLQMNSLRAEDTAVYYCARGYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 240 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 5 - protein, 743 aa, synthetic construct
MYRMQLLSCIALSLALVTNSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGLNDIFEAQKIEWHEGSSGSEISEVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 743 aa, synthetic construct
MYRMQLLSCIALSLALVTNSEISEVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 7 - protein, 738 aa, synthetic construct
MYRMQLLSCIALSLALVTNSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGLNDIFEAQKIEWHEGSSGSEISEVQLVESGGGLVQPGGSLRLSCAASGFNIYYYSIHWVRQAPGKGLEWVASIYSSSGYTSYADSVKGRFTISADTSKNTAYLQMNSLRAEDTAVYYCARYPYWYFDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 738 aa, synthetic construct
MYRMQLLSCIALSLALVTNSEISEVQLVESGGGLVQPGGSLRLSCAASGFNIYYYSIHWVRQAPGKGLEWVASIYSSSGYTSYADSVKGRFTISADTSKNTAYLQMNSLRAEDTAVYYCARYPYWYFDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 9 - protein, 733 aa, synthetic construct
MYRMQLLSCIALSLALVTNSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGLNDIFEAQKIEWHEGSSGSEISEVQLVESGGGLVQPGGSLRLSCAASGFNLYYSYIHWVRQAPGKGLEWVASIYPSYGSTYYADSVKGRFTISRDNSKNTLYLQMNSLRAEDTAVYYCARGYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 733 aa, synthetic construct
MYRMQLLSCIALSLALVTNSEISEVQLVESGGGLVQPGGSLRLSCAASGFNLYYSYIHWVRQAPGKGLEWVASIYPSYGSTYYADSVKGRFTISRDNSKNTLYLQMNSLRAEDTAVYYCARGYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 11 - protein, 212 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQAYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQGYSDLITFGQGTKVEIKRTVAAPSVFIFPPSDSQLKSGTASVVCLLNNFYPREAKVQWKVDNALQSGNSQESVTEQDSKDSTYSLRSTLTLSKADYEKHKVYACEVTHQGLSSPVTKSFNRGEC

SEQ ID NO: 13 - protein, 213 aa, synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQSYYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 497 aa, synthetic construct
MYRMQLLSCIALSLALVTNSDIQMTQSPSSLSASVGDRVTITCRASQSVGSALAWYQQKPGKAPKLLIYSASSLYSGVPSRFSGSRSGTDFTLTISSLQPEDFATYYCQQAYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 15 - protein, 499 aa, synthetic construct
MYRMQLLSCIALSLALVTNSDIQMTQSPSSLSASVGDRVTITCRASQSVGSALAWYQQKPGKAPKLLIYSASSLYSGVPSRFSGSRSGTDFTLTISSLQPEDFATYYCQQGYSDLITFGQGTKVEIKRTVAAPSVFIFPPSDSQLKSGTASVVCLLNNFYPREAKVQWKVDNALQSGNSQESVTEQDSKDSTYSLRSTLTLSKADYEKHKVYACEVTHQGLSSPVTKSFNRGECGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 16 - protein, 498 aa, synthetic construct
MYRMQLLSCIALSLALVTNSDIQMTQSPSSLSASVGDRVTITCRASQSVGSALAWYQQKPGKAPKLLIYSASSLYSGVPSRFSGSRSGTDFTLTISSLQPEDFATYYCQQSYYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGLNDIFEAQKIEWHEGSSGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS

SEQ ID NO: 17 - protein, 25 aa, synthetic construct
GGGGSGLNDIFEAQKIEWHEGSSGS